(19) United States
(12) Patent Application Publication
(10) Pub. No.: US 2023/0323464 A1
(43) Pub. Date: Oct. 12, 2023

(54) METHOD FOR PREDICTING PROGNOSIS OF PATIENTS HAVING EARLY BREAST CANCER

(71) Applicant: SEOUL NATIONAL UNIVERSITY R&DB FOUNDATION, Seoul (KR)

(72) Inventors: Young Kee Shin, Seoul (KR); Hannah Lee, Seoul (KR)

(21) Appl. No.: 17/924,832

(22) PCT Filed: May 12, 2021
(86) PCT No.: PCT/KR2021/005971

§ 371 Date: Jun. 15, 2023

(30) Foreign Application Priority Data
May 12, 2020 (KR) 10-2020-0056699

(51) Int. Cl. C12Q 1/6886; C12Q 1/6851; G01N 33/574

(52) U.S. Cl. CPC C12Q 1/6886; C12Q 1/6851; G01N 33/57484; C12Q 2600/158; C12Q 2600/118

(57) ABSTRACT

The present invention relates to a method for predicting the prognosis of abreast cancer patient and, more particularly, to a method for predicting the prognosis of breast cancer by combining immune-related genes. The present invention is applicable to all breast cancer patients regardless of the breast cancer molecular subtype, and in addition, if the immune-related gene combination is used to predict the prognosis of breast cancer, as provided in the present invention, it is possible to predict the prognosis of a breast cancer patient without information on proliferation genes.

### TECHNICAL FIELD

The present application claims priority to Korea Patent Application No. 2020-0056699 filed on May 12, 2020, and the entire specification is used as a reference in the present application.

The present invention relates to a method for predicting the prognosis of patients' breast cancer and, more particularly, to a method for predicting the prognosis of breast cancer by combining immune-related genes.

### BACKGROUND ART

Breast cancer is the most common cancer in women and has the second highest death rate. Risk factors for developing breast cancer comprise race, age and mutations in tumor suppressor genes BRCA-1 and BRCA-2 and p53. Alcohol intake, high-fat diet, lack of exercise, hormones released after exogenous menopause and ionizing radiation also increase the risk of developing breast cancer. Breast cancer is classified into four subtypes: luminal A, luminal B, HER2, and triple negative breast cancer (TNBC) according to the expression status of hormone receptors (e.g., estrogen receptors and progesterone receptors) and human epidermal growth factor receptor 2 (HER2). Each breast cancer subtype has distinct molecular characteristics.

Current methods for treating breast cancer require an additional adjuvant treatment for reducing future recurrence after a tumor removal surgery (e.g., chemotherapy, anti-hormone therapy, targeted therapy and radiation therapy). Since 70 to 80% of patients with early breast cancer have a very low risk of metastasis to other organs, they do not need chemotherapy. However, whether or not metastasis has occurred would not be accurately determined using conventional guidelines for treating breast cancer. Thus, chemotherapy and radiation therapy after a surgery are prescribed to most patients. However, continuous administration of an anti-cancer agent to a patient for whom chemotherapy is not effective may only increase side effects to cause unwanted pain to the patient. Therefore, it is necessary to exactly predict the prognosis of future cancer in patients with early breast cancer, wisely select the most appropriate treatment method at the right time, and prepare for poor prognosis such as metastatic recurrence.

Meanwhile, signals of proliferation and a cell cycle have conventionally been focused as prognostic indicators of breast cancer, and proliferation/cell cycle-regulating genes as markers have been applied to a gene expression-based analysis for predicting prognosis. Representatively, products (e.g., Oncotype DX, MammaPrint, PAM50 and Endopredict) are commercial assays based on complex gene expression profiling techniques for proliferative genes in frozen or formalin-fixed paraffin-embedded (FFPE) samples. However, since each of these commercially-available kits targets limited subtypes of breast cancer, there is a limitation that the kits cannot be widely used for all molecular subtypes of breast cancer. The Oncotype DX, MammaPrint, PAM50 and Endopredict kits mainly target ER+ type of breast cancer. As seen in these commercial kits, they may only predict prognosis of hormone receptor-positive breast cancer subtypes, and commercial kits for hormone receptor-negative breast cancer subtypes do not yet exist. In addition, according to a recent report by Alvarado M D et al. (Non-Patent Document 1), it was reported that PAM50 expression analysis and results in Oncotype DX in patients classified as at-risk were inconsistent with each other.

Given the current situation, in order to more accurately predict a patient's survival outcome and response to adjuvant chemotherapy, conventional analysis methods used for predicting the prognosis of breast cancer need to be improved, and prognostic analysis methods applicable to various types of breast cancer are required.

### PRIOR ART CITATIONS

### Non-Patent Citations

(Non-Patent Document 1) Alvarado M. D. et al., “A Prospective Comparison of the 21-Gene Recurrence Score and the PAM50-Based Prosigna in Estrogen Receptor-Positive Early-Stage Breast Cancer,” Adv. Ther. (2015), 32:1237-47, doi:10.1007/s12325-015-0269-2

### SUMMARY OF INVENTION

### Technical Problem

The inventors of the present invention have made diligent efforts to find a method for significantly predicting prognosis for all breast cancer molecular subtypes and genetic markers, while breaking away from conventional methods performed mainly on proliferation-related gene analysis to predict the prognosis of cancer. As a result, as described in the present application, the inventors have completed the present invention by confirming that prognosis for all molecular subtypes of breast cancer may be predicted with high accuracy by using a combination of immune-related genes, without information on proliferation genes.

Accordingly, an object of the present invention is to provide a method for predicting the prognosis of breast cancer comprising following steps, in order to provide information necessary for predicting the prognosis of a patient's breast cancer:
(a) measuring the expression levels of immune-related genes from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

Another object of the present invention is to provide a method for predicting the prognosis of breast cancer comprising following steps, in order to provide information necessary for predicting the prognosis of a patient's breast cancer:
(a) measuring the expression levels of the immune-related genes having the group consisting of TRAT1, IL21R, IGHM, CTLA4 and IL2RB, or the group consisting of TRAT1, IL21R and CTLA4 from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

Another object of the present invention is to provide a method for calculating a breast cancer prognostic risk score comprising following steps, in order to provide information necessary for predicting the prognosis of a patient's breast cancer:
(i) measuring the mRNA expression levels of TRAT1, IL21R, IGHM, CTLA4 and IL2RB genes from a biological sample obtained from a patient with breast cancer and the value of LN of the patient with breast cancer;
(ii) standardizing the mRNA expression levels of the genes; and
(iii) calculating a breast cancer prognostic risk score by substituting the standardized value of step (ii) and the value of LN of step (i) into the following formula 2-1:

risk score={(β_{TRAT1}*χ_{TRAT1})+(β_{IL21R}*χ_{IL21R})+(β_{IGHM}*χ_{IGHM})+(β_{CTLA4}*χ_{CTLA4})+(β_{IL2RB}*χ_{IL2RB})}+F*2*LN <Formula 2-1>

risk score={(β_{TRAT1}*χ_{TRAT1})+(β_{IL21R}*χ_{IL21R})+(β_{CTLA4}*χ_{CTLA4})+F*2*LN <Formula 2-2>

Another object of the present invention is to provide a composition for predicting the prognosis of patients' breast cancer, comprising a preparation for measuring the expression amounts of (i) TRAT1, IL21R, IGHM, CTLA4 and IL2RB genes; or (ii) TRAT1, IL21R and CTLA4 genes, and a kit comprising the composition.

Another object of the present invention is to provide a composition for predicting the prognosis of patients' breast cancer, consisting of a preparation for measuring the expression amounts of (i) TRAT1, IL21R, IGHM, CTLA4 and IL2RB genes; or (ii) TRAT1, IL21R and CTLA4 genes, and a kit comprising the composition.

Another object of the present invention is to provide a composition for predicting the prognosis of patients' breast cancer, essentially consisting of a preparation for measuring the expression amounts of (i) TRAT1, IL21R, IGHM, CTLA4 and IL2RB genes; or (ii) TRAT1, IL21R and CTLA4 genes, and a kit comprising the composition.

Another object of the present invention is to provide use of a preparation for measuring the expression amounts of (i) TRAT1, IL21R, IGHM, CTLA4 and IL2RB genes; or (ii) TRAT1, IL21R and CTLA4 genes to prepare an agent for predicting the prognosis of patients' breast cancer.

### Solution to Problem

In order to achieve the object of the present invention, the present invention provides a method for predicting the prognosis of breast cancer comprising following steps to provide information necessary for predicting the prognosis of a patient's breast cancer:
(a) measuring the expression levels of immune-related genes from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

In order to achieve another object of the present invention, the present invention provides a method for predicting the prognosis of breast cancer comprising following steps to provide information necessary for predicting the prognosis of a patient's breast cancer:
(a) measuring the expression levels of the immune-related genes having the group consisting of TRAT1, IL21R, IGHM, CTLA4 and IL2RB, or the group consisting of TRAT1, IL21R and CTLA4 from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

In order to achieve another object of the present invention, the present invention provides a method for calculating a breast cancer prognostic risk score comprising following steps to provide information necessary for predicting the prognosis of a patient's breast cancer:
(i) measuring the mRNA expression levels of TRAT1, IL21R, IGHM, CTLA4 and IL2RB genes from a biological sample obtained from a patient with breast cancer and the value of LN of the patient with breast cancer;
(ii) standardizing the mRNA expression levels of the genes; and
(iii) calculating a breast cancer prognostic risk score by substituting the standardized value of step (ii) and the value of LN of step (i) into the following formula 2-1:

risk score={(β_{TRAT1}*χ_{TRAT1})+(β_{IL21R}*χ_{IL21R})+(β_{IGHM}*χ_{IGHM})+(β_{CTLA4}*χ_{CTLA4})+(β_{IL2RB}*χ_{IL2RB})}+F*2*LN <Formula 2-1>

risk score={(β_{TRAT1}*χ_{TRAT1})+(β_{IL21R}*χ_{IL21R})+(β_{CTLA4}*χ_{CTLA4})+F*2*LN <Formula 2-2>

In order to achieve another object of the present invention, the present invention provides a composition for predicting the prognosis of patients' breast cancer, comprising a preparation for measuring the expression amounts of (i) TRAT1, IL21R, IGHM, CTLA4 and IL2RB genes; or (ii) TRAT1, IL21R and CTLA4 genes, and a kit comprising the composition.

The present invention also provides a composition for predicting the prognosis of patients' breast cancer, consisting of a preparation for measuring the expression amounts of (i) TRAT1, IL21R, IGHM, CTLA4 and IL2RB genes; or (ii) TRAT1, IL21R and CTLA4 genes, and a kit comprising the composition.

The present invention also provides a composition for predicting the prognosis of patients' breast cancer, essentially consisting of a preparation for measuring the expression amounts of (i) TRAT1, IL21R, IGHM, CTLA4 and IL2RB genes; or (ii) TRAT1, IL21R and CTLA4 genes, and a kit comprising the composition.

The present invention also provides use of a preparation for measuring the expression amounts of (i) TRAT1, IL21R, IGHM, CTLA4 and IL2RB genes; or (ii) TRAT1, IL21R and CTLA4 genes to prepare an agent for predicting the prognosis of patients' breast cancer.

Hereinafter, the present invention will be described in detail.

In the present invention, the term “prognosis” refers to the progress of the disease during or after the treatment of breast cancer and preferably to the progress of the disease after the treatment, but the present invention is not limited thereto. In addition, the term “progress of the disease” is a concept including cure, recurrence, metastasis or metastatic recurrence of cancer, and most preferably refers to metastatic recurrence, but the present invention is not limited thereto. Among them, the prediction of the prognosis of metastatic recurrence (or the diagnosis of the prognosis) may determine in advance whether or not the tumor may develop into metastatic breast cancer in the future especially in patients with early breast cancer to provide clues to the direction of the treatment of breast cancer and, thus, is regarded as a very meaningful work.

The term “metastatic recurrence” in the present invention means the transformation of cancer derived from at least one breast tumor after treatment, that is, the separation of cancer cells from the tumor and their continuous growth into cancer at a site separated from the tumor (hereinafter referred to as “a distant site”). The distant site may be, for example, in at least one lymph node, or the cancer cells may be mobile or fixed, ipsilateral or contralateral to the tumor, or on the collarbone or in the armpit.

Specifically, the metastatic recurrence comprises local metastatic recurrence caused from metastasis to the site of occurrence of breast cancer before treatment and/or to the site within the ipsilateral breast and/or contralateral breast, and distant metastatic recurrence caused from metastasis to a distant site (e.g., lung, liver, bone, lymph node, skin and brain), but the present invention is not limited thereto.

The pathological stages of breast cancer are usually classified according to the TNM system of the American Cancer Society, which evaluates three (3) factors of a tumor size (T), a degree of invasion of the tumor into the lymph nodes (N), and distant metastasis to other organs (M). The pathological characteristics in each pathological stage are summarized in Table 1 below.

TABLE 1
Pathological classification of breast
cancer according to TNM stages

| Classification | Specific classification |
|---|---|
| T stages | T0: No evidence of tumor |
| | Tis: Carcinoma in situ |
| | T1: Tumor with the largest diameter of 2 |
| | cm or less |
| | T2: Tumor with the largest diameter of |
| | greater than 2 cm but less than 5 cm |
| | T3: Tumor with the largest diameter of |
| | greater than 5 cm |
| N stages | N0: No metastasis to lymph nodes |
| | N1: Metastasis in one to three lymph nodes |
| | N2: Metastasis in four to nine lymph nodes |
| | N3: Metastasis in ten or more lymph nodes |
| M stages | M0: No distant metastasis |
| | M1: Distant metastasis |

The prognosis may differ even among patients classified into a single stage according to the TNM system and is influenced by breast cancer molecular subtypes. That is, it is known that the condition and prognosis of breast cancer at a single stage may significantly vary depending on the expression status of hormone receptors (e.g., estrogen receptors and progesterone receptors) and HER2. The characteristics of receptors for each breast cancer molecular subtype are as shown in Table 2 below, and it is being reported that the results and prognosis of treatment may vary depending on the breast cancer subtype and, thus, are used as an index for selecting a surgical method or a chemotherapy method.

TABLE 2
Classification of molecular biological subtypes of breast cancer

| | | Frequency |
|---|---|---|
| | | of |
| | | occurrence |
| Subtype | Properties | (%) |
| Luminal A | * ER positive and/or PR positive | 30 to 70 |
| (HR+/HER2−) | * HER2 negative | |
| | * Low expression of Ki67 | |
| Luminal B | * ER positive and/or PR positive | 10 to 20 |
| (HR+/HER2+) | * HER2 positive (or HER2 | |
| | negative while showing high | |
| | expression of Ki67) | |
| TNBC | * ER negative | 15 to 20 |
| (Triple | * PR negative | |
| negative, | * HER2 negative | |
| HR−/HER2−) | | |
| HER2 | * ER negative | 5 to 15 |
| (HR−/HER2+) | * PR negative | |
| | * HER2 positive | |

* HR: a hormone receptor
* ER: an estrogen receptor
* PR: a progesteron receptor
* HR+: comprising ER+/PR−, ER−/PR+ and ER+/PR+

As it has been reported that proliferation/cell cycle-regulating genes are strongly associated with the progression of cancer and have an impact on patients' survival, conventional commercially available kits (e.g., Oncotype DX, MammaPrint, PAM50 and Endopredict) are being used to predict the prognosis of breast cancer. However, they only target ER+ among the types of breast cancer.

On the other hand, the inventors of the present invention first identified that the prognosis of breast cancer may be predicted only with the information on the expression of immune-related genes without using proliferation/cell cycle-regulating genes. In particular, the present invention has technical significance in that the method for predicting the prognosis of breast cancer of the present invention is applicable to all molecular subtypes of breast cancer (i.e., HR+/HER2−, HR+/HER2+, HR−/HER2+ and TNBC).

The present invention provides a method for predicting the prognosis of breast cancer comprising following steps to provide information necessary for predicting the prognosis of a patient's breast cancer:
(a) measuring the expression levels of immune-related genes from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

The biological sample in step (a) in the present invention may preferably be the breast cancer tissue of a patient with breast cancer, but the present is not limited thereto. The breast cancer tissue may comprise some normal cells and be preferably selected from the group consisting of a formalin-fixed paraffin-embedded (FFPE) sample of breast cancer tissue containing the patient's cancer cells, a fresh tissue and a frozen tissue, but the present invention is not limited thereto.

The immune-related genes to be measured in step (b) may preferably be at least two selected from the group consisting of TRBV20-1, CCL19, CD52, SRGN, CD3D, IGJ, HLA-DRA, LOC91316, IGF1, CYBRD1, TMC5, ALDH1A1, OGN, PDCD4, FRZB, CX3CR1, IGFBP6, GLA, LOC96610, IGLL3, ITPR1, SERPINA1, EPHX2, MFAP4, RNASET2, CCNG1, FBLN5, SORBS2, CCBL2, BTN3A2, TFAP2B, LTF, ITM2A, HLA-DPB1, HLA-DMA, RPL3, LOC100130100, FAM129A, ELOVL5, GBP2, RARRES3, GOLM1, RTN1, ICAM3, LAMA2, CXCL13, ZCCHC24, CD37, VTCN1, PYCARD, CORO1A, SH3BGRL, TPSAB1, TNFSF10, ACSF2, TGFBR2, DUSP4, ARHGDIB, TMPRSS3, DCN, LRIG1, FMOD, ZNF423, SQRDL, TPST2, CD44, MREG, GIMAP6, GJA1, IFITM3, BTG2, PIP, RPS9, HLA-DPA1, IMPDH2, TNFRSF17, C14orf139, SPRY2, XBP1, THYN1, APOD, C10orf116, VAV3, FAS, MYBPC1, CFB, TRIM22, ARID5B, PTGDS, TGFBR3, TNFAIP8, SEMA3C, TMEM135, ARHGEF3, PTGER4, ABCA8, ICAM2, HLA-DQB1, HSPA2, CD27, ARMCX1, POU2AF1, IGBP1, PDE4B, ADH1B, WLS, SUCLG2, PGR, STARD13, SORL1, ATP1B1, IFT46, SIK3, LIPT1, OMD, HBB, C3, FGL2, PECI, RAC2, PDZRN3, CXCL12, DPYD, TXNDC15, STOM, EMCN, SCGB2A2, FAM176B, HIGD1A, ACSL5, RPS24, RGS10, RAI2, CNN3, FBXW4, SEPP1, SLC44A4, MGP, ABCD3, SETBP1, APOBEC3G, LCP2, HLA-DRB1, SCUBE2, DEPDC6, RPL15, SH3BP4, MSX2, CLU, DPT, ZNF238, HBP1, GSTK1, ZBTB16, CCDC69, ALDH2, SLC1A1, ARMCX2, HMGCS2, TSPAN3, FTO, PON2, C16orf62, QDPR, LRP2, PSMB8, HCLS1, FXYD1, OAT, SLC38A1, MAOA, LPL, C10orf57, SPARCL1, ERAP2, PDGFRL, RBP4, LRRC17, LHFP, BLNK, HBA2, CST7, TRAT1, IL21R, IGHM, CTLA4, IL2RB, TNFRSF9, CTSW, CCR10, GPR18, CR2, DOCK10, GZMB, ITK, LTB, IGLJ3, IGLV1-44, AIM2, CXCL9, KIAA0125, IL2RG, CD69, CD55, TRAF3IP3, EVI2B, STAP1, KLRB1, PRKCB, GPR171, PPP1R16B, SH2D1A, TNFRSF1B, CD48, BANK1, LY9, VNN2, TCL1A, CYTIP, PTPRC, PDCD1LG2, LTA, IGHG1, and CD19. However, the present invention is not limited thereto. Each gene sequence and sequences of mRNA and the protein therefrom are well known in the art through GenBank, etc. Preferably, the immune-related genes may be a combination of three (3) to twenty (20) genes, and may be a combination of TRAT1, IL21R, IGHM, CTLA4, and IL2RB, or a combination of TRAT1, IL21R and CTLA4.

The types of the immune response-related genes to be measured in step (a) may be selected through Lasso regression analysis. In a more preferred embodiment, the immune response-related genes to be measured in step (a) may be selected by comprehensively considering the results of Cox univariate proportional hazards regression analysis or Cox multivariate proportional hazards regression analysis, in addition to Lasso regression analysis.

In the present invention, the expression “measuring the expression levels of the genes” means detection of the expression levels of the target genes, more preferably quantitative detection of the expression levels of the target genes and obtaining the quantified expression levels or amounts. The measurement of the expression levels of the target genes may preferably be performed by measuring the mRNA expression levels of the target genes or the expression levels of the proteins encoded by the genes, but the present invention is not limited thereto.

A method for measuring the mRNA expression levels of the genes may be methods using a pair of primers or probes specifically binding to the genes, and these methods are known in the art. For example, a method for measuring the mRNA expression levels of the genes may be one selected from the group consisting of microarrays, polymerase chain reaction (PCR), RT-PCR, quantitative RT-PCR (qRT-PCR), real-time polymerase chain reaction (real-time PCR), northern blot, DNA chips and RNA chips, but the present invention is not limited thereto.

A method for measuring the expression levels of the proteins may be methods using an antibody specific to the proteins, and these methods are known in the art. For example, the analysis method for measuring the expression levels of the proteins may comprise enzyme linked immunosorbent assay (ELISA), FACS, protein chips, etc., but the present invention is not limited thereto.

In one preferred embodiment, the measurement of the expression levels of the genes is the measurement of the expression levels of mRNA of the genes. The types of the methods for measuring the expression levels of mRNA of the genes are not particularly limited as long as they are known in the art as a quantitative measurement method, and the types are as described above.

For the measurement of the expression levels of mRNA (i.e., detection of the expression levels), the isolation of mRNA from the sample tissue and the synthesis process of cDNA from the mRNA may be required. For the isolation of mRNA, a proper method for isolating RNA conventionally known in the art may be used depending on the sample. In a preferred example, the sample handled in the present invention may be an FFPE sample, and, accordingly, a method for isolating mRNA suitable for the FFPE sample may be used in the present invention. The synthesis process of cDNA may comprise a method of synthesizing cDNA using mRNA as a template conventionally known in the art.

In one embodiment, the measurement of the expression levels of the marker (e.g., selected immune-related genes) for predicting the prognosis of breast cancer of the present invention may preferably be performed for the purpose of quantitative detection of the mRNA expression in a FFPE sample, and, accordingly, the measurement by a method for isolating mRNA and a real time reverse transcription quantitative polymerase chain reaction (RT-qPCR) method may be performed for the FFPE sample.

In addition, the measurement of the expression levels of the target genes in the present invention may be performed according to a method commonly known in the art but may also be performed by an optical quantitative analysis system using a probe labeled with a reporter fluorescent dye and/or a quencher fluorescent dye. The measurement may be performed by a system such as commercially available equipment, for example, ABIPRISM 7700™ Sequence Detection System™, Roche Molecular Biochemicals Lightcycler, and a software affiliated therewith. Such measurement data may be expressed as measured values or threshold cycles (e.g., Ct and Cp). The point at which the measured fluorescence value is recorded as statistically significant for the first time is the threshold cycle, which appears in inverse proportion to the initial value at which the target of detection exists as a template for the PCR reaction. As such, when the value of the threshold cycle is small, it indicates that there are quantitatively more targets of detection.

Since there may be differences in the overall expression amounts or expression levels of the genes depending on the target patient or sample, the expression levels of the genes measured in step (a) need to be standardized. The standardization is performed by calculating a relative expression value of the target gene with respect to the expression amount or expression level of a gene (i.e, a standard gene) capable of representing a basic expression amount or level. The techniques for standardizing the expression level of a genes are well known in the art.

In one embodiment, the standardization may be calculated, for example, into a relative expression value to the expression amount of a standard expression gene (or a housekeeping gene) known in the art, or by an algorithm for standardization of a data set, such as ComBat algorithm. For example, the standardization is to measure the expression amounts of one to three genes selected from the group consisting of C-terminal-binding protein 1 (CTBP1), cullin 1 (CUL1) and Ubiquilin-1 (UBQLN1) (or, if multiple genes are selected, the average of their expression amounts) and then calculate the relative expression value of the target gene (i.e., the immunity-related gene targeted in the present invention).

Step (c) is a step of predicting the prognosis of breast cancer by combining the expression levels of the immune-related genes standardized in step (b).

In the present invention, the term “poor prognosis” refers to a high-risk group with a high probability of metastasis, recurrence or metastatic recurrence of cancer after treatment, and the term “good prognosis” refers to a low-risk group with a low probability of metastasis, recurrence or metastatic recurrence of cancer.

In a preferred embodiment, the term “poor prognosis” refers to a high-risk group with a high probability of metastasis, recurrence or metastatic recurrence of cancer within 10 years, and the term “good prognosis” refers to a low probability of metastasis, recurrence, or metastatic recurrence of cancer within 10 years. The term “10 years” means 10 years from the time when primary breast cancer is removed from a patient by a surgery (i.e., from the date of the surgery).

In a more preferred embodiment, the term “poor prognosis” refers to a high-risk group with a high probability of metastasis, recurrence or metastatic recurrence of cancer within 5 years, and the term “good prognosis” refers to a low-risk group with a low probability of metastasis, recurrence or metastatic recurrence of cancer within 5 years. The term “5 years” means 5 years from the time when cancer is removed from a patient with primary breast cancer by a surgery (i.e., from the date of surgery).

The method for predicting the prognosis of the present invention is characterized in that, when combining the genes in step (c), proliferation-related genes are excluded. This is significantly different from many conventional methods for predicting the prognosis of cancer based on a strong association between proliferation genes and the onset/progression of cancer. In the present invention, the prognosis of breast cancer may be more accurately predicted by combining the immune-related genes selected in step (a), and the overexpression of the combined immune-related genes is closely correlated with good prognosis of breast cancer.

The method for predicting the prognosis of breast cancer using immune-related genes of the present invention is used to predict the risk of recurrence or distant metastasis after a breast cancer surgery, and such predictive information may also be used to predict a patient's response to adjuvant chemotherapy. That is, the present invention may be used to select patients who do not require additional chemotherapy after a surgery for primary breast cancer. Therefore, the patients' breast cancer to be sampled in step (a) are preferably those who do not receive any chemotherapy before and after surgery. Among the patients who do not receive any chemotherapy, the patient group predicted to have good prognosis according to the present invention has a low probability of metastasis, recurrence or metastatic recurrence within 10 years and, thus, does not require additional chemotherapy after the surgery. However, since the group with poor prognosis has a high probability of metastasis, recurrence or metastatic recurrence within 10 years after the surgery, additional chemotherapy after the surgery may be recommended to them.

In addition, the method for predicting the prognosis of the present invention may additionally comprises a step of evaluating the presence of LN according to the TNM system, and is characterized in that LN in step (c) predicts poor prognosis when the cancer has metastasized to the lymph nodes. That is, the prognosis of breast cancer may be accurately predicted by combining the immune-related genes and metastasis status to the lymph nodes (LN). The method for predicting the prognosis of breast cancer by such combination has not been previously reported. That is, according to the above-described method, the prognosis of breast cancer may be more accurately predicted by determining the expression levels of the genes measured in step (a) and the presence of LN as the factors for predicting the prognosis of breast cancer.

In the present invention, the LN refers to a method for determining whether metastasis to lymph nodes is caused, by using pathological classification among methods for classifying the stages of breast cancer. The pathological classification, also called postsurgical histopathological classification, is a method of classifying the pathological stages by combining information obtained from patients' breast cancer before starting treatment and information obtained from a surgery or pathological examinations. LN is a method of determining the pathological stages based on the degree of metastasis to the lymph nodes among the pathological classification methods. LN classifies the pathological stages into the case that metastasis to lymph nodes has occurred and the case that metastasis to lymph nodes has not occurred. The case that metastasis to lymph nodes has not occurred is indicated as pN-0 stage, as shown in Table 1 above. pN-1 to pN-3 stages indicate the cases that metastasis to lymph nodes has occurred.

In one preferred embodiment, the term “combination of genes” in step (c) may mean a mathematical combination, but the present invention is not limited thereto. That is, in step (c), the expression values of the immune-related genes standardized in step (b) are mathematically combined to calculate a total score, and the total score may indicate the prognosis of a patient's breast cancer. The term “total score” in the present invention contains information about the prognosis of a patient's breast cancer and, thus, is also referred to as a breast cancer prognosis prediction score or a breast cancer prognostic risk score. In particular, the breast cancer prognostic risk score in the present invention contains information on immune genes and, thus, is also referred to as an immune index.

The term “mathematical combination” in the present invention means mathematically combining expression levels, and the expression values of the standardized immune-related genes are applied to a mathematical algorithm to obtain a total numerical value (i.e., a total score).

In one embodiment, the mathematical algorithm may preferably be a linear regression algorithm. As an example of a specific aspect, the mathematical combination may be a linear combination of each expression value of the immune-related genes and a Cox Regression estimate. In this case, when the number of the immune-related genes is n, the mathematical combination may be performed by the following Formula 1:

Total score=(β₁*χ₁)+(β₂*χ₂)+ . . . +(βₙ*χₙ) [Formula 1]

In the above formula, Xn is the expression value of the n^{th} gene, and βₙ is the Cox Regression estimate of the n^{th} gene.

In the specification of the present invention, the symbol of “*” used in a certain formula indicates multiplication.

In another embodiment, the mathematical combination of the standardized expression values of immune-related genes is a mathematical combination additionally comprising the values of LN according to the TNM system. As an example of this embodiment, when the number of immune response-related genes is n, the mathematical combination may be performed according to the following formula 2:

Total score=(β₁*χ₁)+(β₂*χ₂)+ . . . +(βₙ*χₙ)+F*LN [Formula 2]

In the above formula,

χₙ is the expression value of the n^{th} gene,

βₙ is the Cox Regression estimate of the n^{th} gene,

LN is an integer indicating whether or not metastasis to the lymph nodes has occurred (LN is a value determined according to the pathological judgment on metastasis to the lymph nodes, and is indicated as 0 (when no metastasis to the lymph nodes has occurred) or 1 (when metastasis to the lymph nodes has occurred)), and

F is the Cox Regression estimate for LN.

When the mathematical combination method is used as described above, a threshold value is determined for the total score, and the threshold value is compared with the total score. If the total score is greater than the threshold value, the prognosis may be predicted as poor. The threshold value, which is a criterion for judgment in the present invention, is also referred to as a “reference value” in the specification of the present invention, and may be set to one, or two or more.

In one embodiment, by determining one or two threshold values for the total score and comparing the total score against the threshold value, the patients may be classified into high-risk and low-risk groups; or a high-risk group, an intermediate-risk group and a low-risk group.

As a preferred embodiment, the threshold values may be cut-offs of the 97.5 percentile when indicated by the distribution of the normalized total scores obtained from a plurality of patients with breast cancer. In this case, if the total score calculated for any patient with breast cancer is equal to or greater than the cut-off of the 97.5 percentile, the prognosis of breast cancer may be predicted as poor.

As another embodiment, the threshold values may be cut-offs of the 2.5 percentile and cut-offs of the 97.5 quartile when indicated by the distribution of the normalized total scores obtained from a plurality of patients with breast cancer. In this case, the prediction of the prognosis of breast cancer is performed as follows:
(1) If the total score calculated for a patient with breast cancer is below the cut-off of the 2.5 percentile, the patient is predicted as a low-risk group for breast cancer recurrence;
(2) If the total score calculated for a patient with breast cancer is equal to or greater than the cut-off of the 97.5 percentile, the patient is predicted as a high-risk group for breast cancer recurrence; and
(3) If the total score calculated for a patient with breast cancer is between the cut-off of the 2.5 percentile and the cut-off of the 97.5 percentile, the patient is predicted as a medium-risk group for breast cancer recurrence.

The normalization is not particularly limited as long as it is performed by a statistical processing method known in the art, but may be preferably performed by a bootstrapping method as an example.

As an example of a preferred embodiment, the present invention provides a method of using immune-related genes consisting of TRAT1, IL21R, IGHM, CTLA4 and IL2RB as markers for predicting the prognosis of breast cancer in steps (a) and (c). As such, the present invention provides a method of predicting the prognosis of breast cancer, comprising following steps in order to provide information necessary for predicting the prognosis of a patient's breast cancer:
(a) measuring the expression levels of the immune-related genes having the group consisting of T Cell Receptor Associated Transmembrane Adaptor 1 (TRAT1), Interleukin 21 Receptor (IL21R), Immunoglobulin Heavy Constant Mu (IGHM), Cytotoxic T-Lymphocyte Associated Protein 4 (CTLA4) and Interleukin 2 Receptor Subunit Beta (IL2RB) from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

In addition, the present invention provides a method of using immune-related genes consisting of TRAT1, IL21R and CTLA4 as markers for predicting the prognosis of breast cancer in steps (a) and (c). As such, the present invention provides a method of predicting the prognosis of breast cancer comprising following steps, in order to provide information necessary for predicting the prognosis of a patient's breast cancer:
(a) measuring the expression levels of the immune-related genes having the group consisting of T Cell Receptor Associated Transmembrane Adaptor 1 (TRAT1), Interleukin 21 Receptor (IL21R) and Cytotoxic T-Lymphocyte Associated Protein 4 (CTLA4) from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

The combination of the group consisting of TRAT1, IL21R, IGHM, CTLA4 and IL2RB, or the group consisting of TRAT1, IL21R and CTLA4 as markers may be effective especially for predicting the prognosis of early breast cancer.

In an embodiment for combining TRAT1, IL21R, IGHM, CTLA4 and IL2RB as markers, steps (a) to (c) may be preferably performed by the gene combination of the following (i) or (ii):
(i) TRAT1, IL21R, IGHM, CTLA4 and IL2RB; or
(ii) TRAT1, IL21R and CTLA4.

The embodiments of the specification of the present invention have confirmed that, when the gene combination of (i) or (ii) is used as a variable for predicting the prognosis of breast cancer, the patients may be significantly classified into high-risk and low-risk groups for recurrence of all breast cancer molecular subtypes (i.e., HR+/HER2−, HR+/HER2+, HR−/HER2+ and TNBC).

With regard to the combinations of immune-related genes such as (i) or (ii), although it was previously known that high expression of IL21R and CTLA4, respectively, negatively affects breast cancer survival results, the combination thereof with other immune genes provided by the present invention and their overexpression result in good prognosis as the results of prediction. As such, the method for predicting the prognosis of breast cancer of the present invention has technical specificity.

The sequence of each gene, mRNA nucleotide sequence therefrom, and amino acid sequence of the protein in a human being are well known in the art, for example, through NCBI GenBank. The information of TRAT1 (Gene ID: 50852), IL21R (Gene ID: 50615), IGHM (Gene ID: 3507), CTLA4 (Gene ID: 1493), and IL2RB (Gene ID: 3560) published in NCBI GenBank may be used as a reference.

The embodiment for combining TRAT1, IL21R, IGHM, CTLA4, and IL2RB as markers may additionally comprise a step of evaluating the presence of LN according to the TNM system, and is characterized in that, in the step (c), when metastasis to the lymph nodes occurs, the prognosis is predicted as poor. In addition, in this embodiment, the “combination of genes” in step (c) may more preferably be a mathematical combination, and the specific explanation therefor is as described above.

As a more preferred embodiment of the present invention by the mathematical combination, the present invention provides a method for calculating a breast cancer prognostic risk score comprising following steps, in order to provide information necessary for predicting the prognosis of a patient's breast cancer:
(i) measuring the mRNA expression levels of TRAT1, IL21R, IGHM, CTLA4 and IL2RB genes from a biological sample obtained from a patient with breast cancer and the value of LN of the patient with breast cancer;
(ii) standardizing the mRNA expression levels of the genes; and
(iii) calculating a breast cancer prognostic risk score by substituting the standardized value of step (ii) and the value of LN of step (i) into the following formula 2-1:

risk score={(β_{TRAT1}*χ_{TRAT1})+(β_{IL21R}*χ_{IL21R})+(β_{IGHM}*χ_{IGHM})+(β_{CTLA4}*χ_{CTLA4})+(β_{IL2RB}*χ_{IL2RB})}+F*2*LN <Formula 2-1>

(In formula 2-1, x is the standardized value of the expression levels of the genes indicated by a subscript,

β_{TRAT1} is −0.567144 to −0.1952896, β_{IL21R} is −0.9759746 to −0.3412672, β_{IGHM} is −0.5428339 to −0.1855019, β_{CTLA4} is −0.7454524 to −0.2010003, and β_{IL2RB} is −1.1701.266 to −1.14698,

N is an integer indicating the presence of LN, and

F is from 0.3910642 to 1.013551).

In addition, the present invention provides a method for calculating a breast cancer prognostic risk score, comprising following steps in order to provide information necessary for predicting the prognosis of a patient's breast cancer:
(i) measuring the mRNA expression levels of TRAT1, IL21R and CTLA4 genes from a biological sample obtained from a patient with breast cancer and the value of LN of the patient with breast cancer;
(ii) standardizing the mRNA expression levels of the genes; and
(iii) calculating a breast cancer prognostic risk score by substituting the standardized value of step (ii) and the value of LN of step (i) into the following formula 2-2:

risk score={(β_{TRAT1}*χ_{TRAT1})+(β_{IL21R}*χ_{IL21R})+(β_{CTLA4}*χ_{CTLA4})+F*2*LN <Formula 2-2>

(In formula 2-2, x is the standardized value of the expression levels of the genes indicated by a subscript,

β_{TRAT1} is −1.06659 to −0.2163024, β_{IL21R} is −0.5429339 to −0.01642154, and β_{CTLTA4} is −0.5934638 to −0.1644545,

N is an integer indicating the presence of LN, and

F is from 0.311146 to 0.9303696).

Formulae 2-1 and 2-2 reflect the combination of genes (i) and (ii), respectively, described above. A linear combination of the values of the target genes and LN (i.e., the values of gene expression and the Cox Regression estimates as coefficients) is performed to produce the total score. Since the total score independently has breast cancer prognostic information, the total score is also referred to as a breast cancer prognostic risk score in the specification of the present invention, and the specific explanation therefor is as described above.

The extent to which prognostic predictors (e.g., genes and clinical information) affect the survival rate may be indicated as a quantitative value through Cox proportional hazard analysis. The Cox proportional hazards models express the degree of influence of prognostic factors on the survival rate through a value of the proportional hazard ratio (HR), which is a proportional value of the risk when there is no prognostic factor and the risk when there is a prognostic factor. If the value of the proportional hazard ratio (HR) is greater than 1, the risk when there is a prognostic factor is higher than the risk when there is no prognostic factor, and when the value of the HR is less than 1, the risk when there is a prognostic factor is lower than the risk when there is no prognostic factor. The value obtained by converting the proportional hazard ratio of each prognostic factor to a log scale is called the coefficient of each factor, and this value is used in the present invention as the coefficient of the formula for calculating the breast cancer prognostic risk score (see Cox, David R., “Regression Models and Life-Tables,” Journal of the Royal Statistical Society, Series B (Methodological) (1972): 187-220). With regard to the coefficients of the genes, the validity of the results of the calculation formula was verified through cross validation.

The Cox Regression estimate is also referred to as a “regression coefficient” and is described herein in the form of a “β gene.” That is, βTRAT1, βIL21R, βIGHM, βCTLA4 and βIL2RB refer to the Cox Regression estimates for TRAT1, IL21R, IGHM, CTLA4 and IL2RB, respectively. In the formula of the present invention, each coefficient is applied within the range of a 95% confidence interval of the coefficient value (point estimate) calculated as a result of survival analysis using Cox regression, and a point estimate may preferably be used. The 95% confidence interval values and point estimates of the regression coefficients for each gene and the presence of LN are shown in Table 10.

In the formula, the standardized expression value of the expression level (expression amount) of each gene indicated by a subscript is substituted for the “χ gene.” That is, χ_{TRAT1}, χ_{IL21R}, χ_{IGHM}, χ_{CTLA4}, and χ_{IL2RB} refer to the standardized expression values for TRAT1, IL21R, IGHM, CTLA4 and IL2RB, respectively. The method of standardizing the expression level (expression amount) is as described above.

In one embodiment, a breast cancer prognostic risk score may be compared with a threshold value, and, when the risk score is greater than the threshold value, prognosis may be predicted as poor. The description of such a threshold is as described above.

As an example of this embodiment, the threshold value is the cut-off of the 97.5 percentile when indicated by the distribution of the normalized breast cancer prognostic risk scores obtained from a plurality of patients' breast cancer, and the cut-off of the 97.5 percentile (percentile) may preferably be −7.1.

In the examples of the specification, −7.1, which is a value obtained by rounding the actually calculated cut-off of the 97.5 percentile to the second decimal place, was set as the threshold (reference value), and it was confirmed that patients with breast cancer whose scores were calculated to be −7.1 or more could be predicted as a recurrence-high-risk group, and patients with breast cancer whose scores were calculated to be less than −7.1 could be predicted as a recurrence-low-risk group (see Example 5).

In another embodiment, the threshold value may be the cut-off of the 2.5 percentile and the cut-off of the 97.5 percentile when indicated by the distribution of the normalized breast cancer prognostic risk scores obtained from a plurality of patients with breast cancer. Preferably, the cut-off of the 2.5 percentile may be −9.4, and the cut-off of the 97.5 percentile may be −7.1.

In the examples of the present invention, −7.1, which is a value obtained by rounding the actually calculated cut-off of the 97.5 percentile to the second decimal place, and −9.4, which is a value obtained by rounding off the actually calculated cut-off of the 2.5 percentile to the second decimal place, were employed as the thresholds (reference values) to confirm that patients with breast cancer having a risk score of −9.4 or less could be predicted as the recurrence-low risk group, patients with breast cancer having a risk score of −7.1 or more could be predicted as the recurrence-high group, and patients with breast cancer having a risk score between −9.4 and −7.1 could be predicted as the intermediate-risk group for recurrence (see Examples 4 and 5).

According to the present invention, a high level of sensitivity and specificity of prognosis prediction may be achieved. The term “sensitivity” refers to a ratio of cases that patients were determined as the high-risk group in the results of the test (prognosis prediction) according to the present invention among patients with recurrence (metastasis) within 10 years, and the term “specificity” refers to a ratio of cases that patients were determined as the low-risk group in the results of the test (prognosis prediction) according to the present invention among patients with non-recurrence (non-metastasis) for 10 years.

The method for predicting the prognosis of breast cancer of the present invention may be used to select patients who do not require additional chemotherapy after a surgery on primary breast cancer. The target patient group of the algorithm of the present invention is preferably a patient group that has not received any chemotherapy before and after a surgery, and a patient group with “good prognosis” has a low probability of metastasis, recurrence or metastatic recurrence within 10 years after the surgery and, thus, may not require chemotherapy. However, the group with “poor prognosis” has a high probability of metastasis, recurrence or metastatic recurrence within 10 years after the surgery, and, thus, additional chemotherapy after the surgery may be recommended to them.

In particular, the algorithm for predicting the prognosis of breast cancer represented by Formula 2-1 or Formula 2-2 was calculated by analyzing immune-related genes and clinical information (the presence of LN) for a wide range of clinical samples, and is very excellent in that it shows greater predictive power for the prognosis of breast cancer than conventional techniques of evaluating the prognosis based on clinical information, which is well shown in Example 6. As shown in Example 6, as a result of comparing the c-index of the risk score model of the present invention and other techniques of predicting the prognosis based on clinical information, it was confirmed that the risk score model of the present invention exhibited significantly high predictive power for the prognosis of breast cancer.

The present invention also provides a composition for predicting the prognosis of patients' breast cancer, comprising a preparation for measuring the expression amounts of:
(i) TRAT1, IL21R, IGHM, CTLA4 and IL2RB genes; or
(ii) TRAT1, IL21R and CTLA4 genes, and
a kit comprising the composition.

The composition and kit may additionally comprise a preparation for measuring the expression amount (expression level) of a standard expression gene known in the art for use in the standardization of the expression amounts (expression levels) of genes.

In one embodiment, the preparation for measuring the expression amounts of the genes may be the preparation for measuring the expression levels of mRNA of the genes; or a preparation for measuring the expression levels of the proteins encoded by the genes, but the present invention is not limited thereto.

In a preferred embodiment, the preparation for measuring the expression levels of mRNA of the genes is a pair of primers and/or probes specifically binding to the genes.

As used herein, the term “primer” refers to an oligonucleotide, and may act as an initiation point for synthesis under the conditions for inducing synthesis of a primer extension product complementary to a nucleic acid chain (a template), that is, conditions (e.g., the presence of nucleotides and a polymerizing agent such as DNA polymerase, and a suitable temperature and pH). Preferably, the primer is a deoxyribonucleotide and a single chain. The primer used in the present invention may comprise naturally occurring dNMP (i.e., dAMP, dGMP, dCMP and dTMP), modified nucleotides or non-natural nucleotides. In addition, the primer may also comprise ribonucleotides.

The primer of the present invention may be an extension primer that is annealed to a target nucleic acid to form a sequence complementary to the target nucleic acid by a template-dependent nucleic acid polymerase, which is extended to a position where the immobilized probe is annealed to occupy the part where the probe is annealed.

The extension primer used in the present invention comprises a hybrid nucleotide sequence complementary to the first position of the target nucleic acid. The term “complementary” means that a primer or probe is sufficiently complementary to selectively hybridize to a target nucleic acid sequence under certain annealing or hybridization conditions, encompasses the meaning of being substantially complementary and perfectly complementary, and preferably means being perfectly complementary. In the specification of the present invention, the term “substantially complementary sequence” used for a primer sequence refers not only to a completely identical sequence, but also to a sequence that is partially matched to a sequence to be compared, within a range that the primer may function by annealing to a specific sequence.

The primer must be long enough to prime the synthesis of the extension product in the presence of a polymerization agent. The suitable length of the primer depends on a number of factors, such as a temperature, a field of application and a source of the primer, but the primer is typically 15 to 30 nucleotides. Short primer molecules generally require lower temperatures to form a sufficiently stable hybrid complex with the template. The term “annealing” or “priming” refers to the apposition of an oligodeoxynucleotide or a nucleic acid to a template nucleic acid, wherein the apposition causes the polymerase to polymerize the nucleotide to form a nucleic acid molecule complementary to the template nucleic acid or a portion thereof.

The sequence of the primer does not need to have a sequence completely complementary to a part of the sequence of the template, and it is sufficient for the sequence to have sufficient complementarity within a range in which the primer may perform its own function by hybridizing with the template. Therefore, the primer in the present invention does not have to have a sequence perfectly complementary to the above-described nucleotide sequence as a template, and it is sufficient for the primer to have sufficient complementarity within the range in which it can hybridize to this gene sequence and act as a primer. The design of such a primer may be easily made by those skilled in the art in view of the above-described nucleotide sequences, for example, using a primer design program (e.g., PRIMER 3 program).

In the present invention, the kit may comprise tools and auxiliary reagents used for other measurements, in addition to a preparation for measuring the expression levels of target genes. The kit comprises components of specific reagents and tools according to the measurement preparation and measurement method, and the measurement method is as described above. As a preferred example, the kit may be a RT-PCR kit, a real-time RT-PCR kit, a real-time QRT-PCR kit, a microarray chip kit, or a protein chip kit.

In one embodiment, the kit may additionally comprise tools, devices, and/or reagents for PCR reaction, isolation of RNA from a sample, and synthesis of cDNA conventionally known in the art, in addition to a pair of primers capable of PCR amplification for each gene. The kit of the present invention may additionally comprise, if necessary, a tube to be used for mixing each component, a well plate, and instructions describing how to use the kit.

In addition, the present invention provides use of the preparation for measuring the expression amounts of (i) TRAT1, IL21R, IGHM, CTLA4 and IL2RB genes; or (ii) TRAT1, IL21R and CTLA4 genes to produce a preparation for predicting the prognosis of patients' breast cancer.

In the specification of the present invention, the term “comprising” is used in the same meaning as “including” or “characterized by,” and does not exclude additional components or method steps not specified for the composition or method according to the present invention. In addition, the term “consisting of” means excluding additional elements, steps or components not individually described. The term “essentially consisting of” means that, in the scope of a composition or method, materials or steps that do not substantially affect the basic characteristics thereof may be contained, in addition to the described materials or steps.

### Advantageous Effects of Invention

The present invention may not only be applied to all patients with breast cancer regardless of breast cancer molecular subtypes, but also predict the prognosis of patients' breast cancer without information on proliferation genes by using a combination of immune-related genes to predict the prognosis of breast cancer according to the present invention.

### BRIEF DESCRIPTION OF DRAWINGS

FIG. 1 shows the overall workflow for the development of new technology for predicting the prognosis of breast cancer using immune genes in the present invention.

FIG. 2 shows a summary of information on cohorts used in the discovery set and validation set, showing the GEO number, total number of patient samples in the data set, survival types (e.g., DFS, DMFS and OS), treatment types, etc.

FIG. 3 shows the results of classifying patients into the molecular subtypes of breast cancer according to clinical risk evaluation criteria (based on Adjuvant! Online) and proliferation gene-risk stratification criteria. As shown in FIG. 3, patients were first divided into four groups according to clinical risk assessment criteria (based on Adjuvant! Online) and proliferation gene-risk stratification criteria, and then groups with similar survival rates to the regression analysis results were combined again. Accordingly, one to three subgroups (i.e., AH, AI and AL) were generated for each subtype.

FIG. 4 shows a Kaplan-Meier plot for DFS/DMFS of each subgroup of AH, AI and AL in the HR+/HER2− subtype.

FIG. 5 shows a Kaplan-Meier plot for DFS/DMFS of each subgroup of AH and AL in the HR+/HER2+ subtype.

FIG. 6 shows a Kaplan-Meier plot for DFS/DMFS of the HR−/HER+ subtype group, and all of the HR−/HER+ subtype groups were classified into the AH group.

FIG. 7 shows a Kaplan-Meier plot for DFS/DMFS of each subgroup of AH and AL in the TNBC (HR−/HER−) subtype.

FIG. 8 shows two optimal cut-off points (i.e., cutoff-1 and cutoff-2) as criteria for predicting prognosis in the prognostic risk score model using immune genes of the present invention.

FIG. 9 shows the Kaplan-Meier curves for DFS/DMFS of the high-risk group and low-risk group classified according to the cutoff-1 criterion using the risk score according to the present invention.

FIG. 10 shows Kaplan-Meier curves for DFS/DMFS of the high-risk group and low-risk group classified according to the cutoff-2 criterion using the risk score according to the present invention.

FIG. 11 shows Kaplan-Meier curves for DFS/DMFS of the high-risk group, immune intermediate-risk group and immune low-risk group classified according to both cutoff-1 and cutoff-2 as criteria using the risk score according to the present invention.

FIG. 12 shows FIG. 11 to which Kaplan-Meier curves for DFS/DMFS of the AL and AI groups (the subgroups classified according to clinical risk evaluation criteria and proliferation gene-risk stratification criteria) are added.

FIG. 13 shows Kaplan-Meier curves for DFS/DMFS of the high-risk group, the intermediate-risk group and the low-risk group in the HR+/HER2− subtype classified using the risk score model of the present invention. For comparison, FIG. 13 additionally shows Kaplan-Meier curves for DFS/DMFS of the AL and AI groups in the HR+/HER2− subtype (the subgroup classified according to clinical risk assessment criteria and proliferative gene-risk stratification criteria).

FIG. 14 shows Kaplan-Meier curves for DFS/DMFS of the high-risk group, intermediate-risk group and low-risk group in the HR+/HER2+ subtype classified using the risk score model of the present invention. For comparison, FIG. 14 additionally shows Kaplan-Meier curves for DFS/DMFS of the AL and AI groups in the HR+/HER2+ subtype (the subgroup classified according to clinical risk assessment criteria and proliferative gene-risk stratification criteria).

FIG. 15 shows Kaplan-Meier curves for DFS/DMFS of the high-risk group, intermediate-risk group and low-risk group of the HR−/HER2+ subtype classified using the risk score model of the present invention. (As described above, the HR−/HER2+ subtype group has been classified into the AH group according to the clinical risk assessment criteria and the proliferation gene-risk stratification criteria).

FIG. 16 shows Kaplan-Meier curves for DFS/DMFS of the immune high-risk group, immune intermediate-risk group and immune low-risk group in the TNBC (HR−/HER2−) subtype classified using the risk score model of the present invention. For comparison, FIG. 16 additionally shows Kaplan-Meier curves for DFS/DMFS of the AL and AI groups in the TNBC (HR−/HER2−) subtype (the subgroup classified according to clinical risk assessment criteria and proliferative gene-risk stratification criteria).

FIG. 17 shows Kaplan-Meier curves for overall survival (OS) of the high-risk group and the low-risk group of the Affymetrix microarray platform GPL96 classified using the risk score model of the present invention.

FIG. 18 shows Kaplan-Meier curves for overall survival (OS) of the high-risk group and the low-risk group of Affymetrix microarray platform GPL570 classified using the risk score model of the present invention.

FIG. 19 shows Kaplan-Meier curves for DFS of the high-risk group and the low-risk group of the Molecular Taxonomy of Breast Cancer International Consortium (METABRIC) cohort classified using the risk score model of the present invention.

FIG. 20 shows Kaplan-Meier curves for OS in the high-risk group and the low-risk group in the HR+/HER2− subtype classified using the risk score model of the present invention.

FIG. 21 shows Kaplan-Meier curves for OS of the high-risk group and the low-risk group in the HR+/HER2+ subtype classified using the risk score model of the present invention.

FIG. 22 shows Kaplan-Meier curves for OS of the high-risk group and the low-risk group in the HR−/HER2+ subtype classified using the risk index model of the present invention.

FIG. 23 shows Kaplan-Meier curves for OS of the high-risk group and the low-risk group in the TNBC (HR−/HER2−) subtype classified using the risk index model of the present invention.

FIG. 24 shows the results of comparing the performance of the risk score model of the present invention (also referred to as an immune index, indicated by the immune index in the Fig.) in predicting the prognosis of breast cancer (particularly, DFS/DMFS prediction), to other conventional methods (previously, methods for predicting prognosis only with clinical characteristics), by calculating c-index.

### BEST MODE FOR INVENTION

Hereinafter, the present invention will be described in detail. However, the embodiments described below are only to illustrate the present invention, and the scope of the present invention is not limited to the embodiments described below.

### Example 1: Data Selection of Breast Cancer Patients

A. Discovery set: A Public database, National Center for Biotechnology Information Gene Expression

Omnibus (GEO, http://www.ncbi.nlm.nih.gov/geo), was thoroughly to collect five different breast cancer data for analysis.

The data sets used in this study were strictly selected according to the following criteria: 1) ER (estrogen receptor) status or breast cancer molecular subtype must be confirmed in the clinical data, 2) The patient has not received chemotherapy, 3) The data set has been investigated with the Affymetrix platform ([HG-U133_Plus_2] Affymetrix Human Genome U133 Plus 2.0 Array or [HG-U133A] Affymetrix Human Genome U133A Array),

4) The data set should include survival information, and include DFS/DMFS (disease free survival/distant-metastasis free survival) or OS (overall survival) information as a more desirable endpoint, 5) The data set should include clinical information on lymph node status, tumor size, patient age and histological grade.

Finally, microarray data sets to be used in this study were selected from GSE6532, GSE7390, GSE1121, GSE31519, and GSE4922 cohorts (named a discovery set), and they were all investigated with the same platforms, AffymetrixGPL96. A total of 967 patient samples were analyzed.

The Summary of conventional clinicopathological characteristics for all patients, organized by molecular subtype and cohort, are shown in Table 3 below.

TABLE 3

| | Total | | HR+/HER2− | | HR+/HER2+ | | HR−/HER2- | | HK−/HER2− | |
|---|---|---|---|---|---|---|---|---|---|---|
| | (n = 967) | | (n = 619) | | (n = 99) | | (n = 47) | | (n = 202) | |
| | No. (%) | | No. (%) | | No. (%) | | No. (%) | | No. (%) | |
| Age (years) | | | | | | | | | | |
| <50 | 316 | (32.7) | 160 | (25.8) | 42 | (42.4) | 17 | (36.2) | 97 | (48.0) |
| ≥50 | 651 | (67.3) | 459 | (74.2) | 57 | (57.6) | 30 | (63.8) | 105 | (25.0) |
| Tumor size (cm) | | | | | | | | | | |
| ≤2 | 522 | (54.0) | 321 | (51.9) | 48 | (48.5) | 14 | (29.8) | 139 | (68.8) |
| 2~5 | 432 | (44.7) | 289 | (46.7) | 43 | (48.5) | 33 | (70.2) | 62 | (30.7) |
| >5 | 13 | (1.3) | 9 | (1.4) | 3 | (3) | | | 1 | (0.5) |
| Lymph node status | | | | | | | | | | |
| Negative | 736 | (76.1) | 457 | (73.8) | 75 | (75.8) | 30 | (63.8) | 174 | (86.1) |
| Positive | 181 | (18.7) | 136 | (22.0) | 19 | (19.2) | 11 | (23.4) | 15 | (7.4) |
| NA | 50 | (5.2) | 26 | (4.2) | 5 | (5) | 6 | (12.8) | 13 | (6.4) |
| Histologic grade | | | | | | | | | | |
| 1 | 289 | (29.9) | 164 | (26.5) | 70 | (70.7) | 14 | (29.8) | 41 | (20.3) |
| 2 | 378 | (39.1) | 353 | (57.0) | 0 | (0.0) | 0 | (0.0) | 25 | (12.4) |
| 3 | 299 | (30.9) | 102 | (16.5) | 28 | (28.3) | 33 | (70.2) | 136 | (67.3) |
| NA | 1 | (0.1) | 0 | (0.0) | 1 | (1) | 0 | (0.0) | 0 | (0.0) |

| | Total | | GSE6532 | | GSE7390 | | GSE11121 | | GSE31519 | | GSE4922 | |
|---|---|---|---|---|---|---|---|---|---|---|---|---|
| | (n = 967) | | (n = 256) | | (n = 161) | | (n = 200) | | (n = 105) | | (n = 245) | |
| | No. (%) | | No. (%) | | No. (%) | | No. (%) | | No. (%) | | No. (%) | |
| Age (years) | | | | | | | | | | | | |
| <50 | 316 | (32.7) | 61 | (23.8) | 109 | (67.7) | 47 | (23.5) | 49 | (46.7) | 50 | (2 [symbol] .4) |
| ≥50 | 651 | (67.3) | 195 | (76.2) | 52 | (32.3) | 153 | (76.5) | 56 | (53.3) | 195 | (79. [symbol] ) |
| Tumor size (cm) | | | | | | | | | | | | |
| ≤2 | 522 | (54.0) | 115 | (44.9) | 66 | (41.0) | 112 | (56) | 105 | ( [symbol] 00) | 124 | (50.6) |
| 2~5 | 432 | (44.7) | 137 | (53.5) | 95 | (59.0) | 85 | (42.5) | 0 | (0.0) | 115 | (46.9) |
| >5 | 13 | (1.3) | 4 | (1.6) | 0 | (0.0) | 3 | (1.5) | 0 | (0.0) | 6 | (2.4) |
| Lymph node status | | | | | | | | | | | | |
| Negative | 736 | (76.1) | 176 | (68.8) | 103 | (64.0) | 200 | (100) | 100 | (95.2) | 157 | (64.1) |
| Positive | 181 | (18.7) | 77 | (30) | 18 | (11.2) | 0 | (0.0) | 5 | (4.8) | 81 | (33.1) |
| NA | 50 | (5.1) | 3 | (1.2) | 40 | (24.8) | 0 | (0.0) | 0 | (0.0) | 7 | (2.8) |
| Histologic grade | | | | | | | | | | | | |
| 1 | 289 | (29.9) | 88 | (34.4) | 27 | (16.8) | 55 | (27.5) | 35 | (33.3) | 84 | (34.3) |
| 2 | 378 | (39.1) | 109 | (42.6) | 53 | (32.9) | 110 | (55) | 0 | (0.0) | 106 | (43.3) |
| 3 | 299 | (30.9) | 58 | (22.6) | 81 | (50.3) | 35 | (17.5) | 70 | (66.7) | 55 | (22.4) |
| NA | 1 | (0.1) | 1 | (0.4) | 0 | (0.0) | 0 | (0.0) | 0 | (0.0) | 0 | (0.0) |

[symbol]

indicates data missing or illegible when filed

B. Validation set: As a data set for validation, a microarray data sets was selected from GSE21653, GSE42568, and GSE3494 cohorts. In addition, for further validation using other platforms, METABRIC gene expression profiles were analyzed with the same criteria applied to the microarray data set. Data were downloaded via the cBioportal website (http://www.cbioportal.org/index.do) and log 2 normalized prior to analysis.

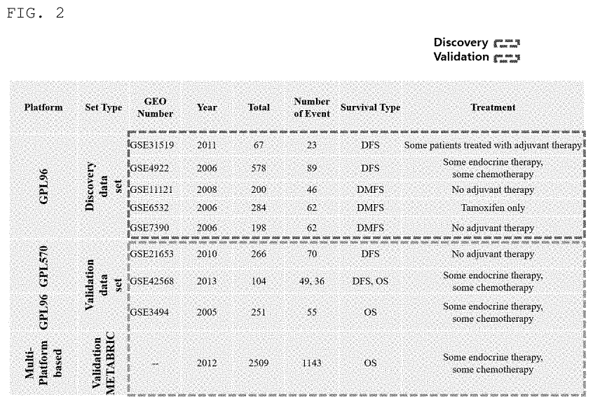

Data sets and platforms used in the discovery set and validation in above were summarized in FIG. 2. The patient data in this study were used those classified as 0 (when no metastasis to lymph nodes has occurred) or 1 (when metastasis to lymph node has occurred) whether breast cancer LN- or not according to the cancer metastasis classification TNM (Tumor Node Metastasis) system.

### Example 2: Risk Stratification of Patient Breast Cancer Prognosis According to the Existing Method Using Proliferation/Cell Cycle Related Genes

2-1. Data Mining

Based on the information that can identify the molecular subtypes of breast cancer, each patient was classified into four subtypes of breast cancer: HR+/HER2−(ER+ or PR+/HER2−), HR+/HER2+(ER+ or PR+/HER2+), HR−/HER2+(ER−/PR−/HER2+), or TNBC (ER−/PR−/HER2−).

Data downloaded in Example 1 were log 2 normalized before analysis. Next, in the discovery set, genes exceeding the threshold of the interquartile range were optionally selected to reduce bias. In addition, in order to reduce the non-biological variation present in the selected data set, batch effect correction was performed on the discovery set and validation set using ComBat algorithm, and verified with principal component analysis. After the correction and normalization were performed, the data of each molecular subtype were stratified into 4 risk categories by clinical data and gene risk classification schemes (see Examples 2-4 blow).

2-2. Survival Analysis

The most preferred endpoint used when performing survival analysis is OS(Overall Survival), but OS information is not always available due to temporal limitations. Therefore, when there is a time limit, DFS (Disease Free Survival) or DMFS (Distant-Metastasis Free Survival) is used as a endpoint instead of OS. In this study, OS (overall Survival) and DFS/DMFS were also used as clinical endpoints. Univariate and multivariate analyzes of clinical and genetic variables were performed using Cox proportional hazard regression analysis.

Multivariate analysis confirmed independent contributions of the predictor variables. In addition, Survival results were graphed using the Kaplan-Meier method and log-rank test, and in differences survival between groups were identified. Statistically significant was estimated when the Log rank p-value<0.05. The above mentioned methods were also used in the same way in the embodiments described later (Examples 3 to 5).

2-3. Gene Ontology and Pathway Analysis

Gene annotation and pathway analysis were performed according to breast cancer subtype. Annotation of gene pathways consists of two parts. First, the most significant genes with p-value of 0.01 or less from DAVID were annotated. For further analysis, using gene annotation package topGO in R version 3.4.3., the pathways of the most significant genes in the regressive analysis were annotated. topGO applies two types of statistics, Fisher's exact test and Kolmogorov-Smirnov test, to calculate gene scores to find the most important pathway. Also, two types of algorithms, the classic method and the elim method can be applied to each statistic.

In this study, the above mentioned two algorithms were applied to the Kolmogorov-Smirnov test, and the classic Fisher was used to find the most important annotations. Prior to pathway analysis, breast cancer types commonly classified into four subtypes were grouped into three groups: total HR−, HR+/HER+, and HR+/HER−. Because of no statistical difference in survival results between HR−/HER+ and HR−/HER− subtypes (data not shown), so they were combined as HR−.

The results of gene annotation and pathway analysis were shown in Table 4 below. Table 4 shown that most of genes significantly contributing to survival in the HR+ type were related to cell proliferation and cell cycle regulation, whereas the genes significantly contributing to survival in the HR− type were related to locomotion and immune response.

TABLE 4

| | | | | | | | |
|---|---|---|---|---|---|---|---|
| GO.ID | Term | Annotated | Significant | Expected | Rank in classicKS | classicKS | elimKS |

HR+/HER2−

| | | | | | | | |
|---|---|---|---|---|---|---|---|
| GO:0030154 | cell differentiation | 54 | 54 | 54 | 175 | 0.77513 | 0.016 |
| GO:0050793 | regulation of developmental process | 40 | 40 | 40 | 107 | 0.32526 | 0.023 |
| GO:0048869 | cellular developmental process | 55 | 55 | 55 | 174 | 0.7526 [symbol] | 0.028 |
| GO:2000026 | regulation of multicellular organi [symbol] al d [symbol] | 35 | 35 | 35 | 113 | 0.33666 | 0.043 |
| GO:0033554 | cellular response to stress | 55 | 55 | 55 | 108 | 0.32814 | 0.044 |
| GO:0051173 | positive regulation of nitrogen compound [symbol] | 57 | 57 | 57 | 36 | 0.02 [symbol] 73 | 0.055 |
| GO:0010604 | positive regulation of m [symbol] | 60 | 60 | 60 | 42 | 0. [symbol] 3029 | 0.093 |
| GO:0031325 | positive regulation of cellular metab [symbol] | 54 | 54 | 54 | 49 | 0.03221 | 0.098 |
| GO:0 [symbol] 42981 | regulation of apop [symbol] process | 31 | 31 | 31 | 168 | 0.71457 | 0.098 |
| GO:0006366 | transcription from RNA polymerase [symbol] pr [symbol] | 38 | 38 | 38 | 95 | 0.24381 | 0.115 |
| GO:0043067 | regulation of programmed cell death | 31 | 31 | 31 | 169 | 0.71457 | 0.14 [symbol] |
| GO:0045935 | positive regulation of nucl [symbol] base [symbol] contai [symbol] | 31 | 31 | 31 | 124 | 0.38235 | 0.151 |

HR+/HER2+

| | | | | | | | |
|---|---|---|---|---|---|---|---|
| GO:0006260 | DNA replication | 38 | 38 | 38 | 7 | 3.50E−05 | 3.50E−05 |
| GO:0044772 | mitotic cell cycle phase trans [symbol] ion | 70 | 70 | 70 | 4 | 7.90E−06 | 0.00071 |
| GO:0000070 | mitotic sister chromatid segregation | 30 | 30 | 30 | 10 | 0.0011 | 0.00111 |
| GO:0051301 | cell division | 71 | 71 | 71 | 12 | 0.0015 | 0.0 [symbol] 152 |
| GO:0000082 | G [symbol] transition of mitotic cell cycle | 34 | 34 | 34 | 15 | 0.0021 | 0.00215 |
| GO:0007346 | regulation of mitotic cell cycle | 63 | 63 | 63 | 19 | 0.0029 | 0.00287 |
| GO:1901987 | regulation of cell cycle phase transitio [symbol] | 49 | 49 | 49 | 22 | 0.00 [symbol] | 0.00805 |
| GO:00 [symbol] 0068 | positive regulation of cell cycle proces [symbol] | 33 | 33 | 33 | 23 | 0.0081 | 0.008 [symbol] 7 |
| GO:190 [symbol] 647 | mitotic cell cycle process | 110 | 110 | 110 | 2 | 4.30E−07 | 0.00858 |
| GO:0006974 | cellular response to DNA damage stimulus | 71 | 71 | 71 | 25 | 0.011 | 0.01104 |
| GO:19 [symbol] 1990 | regulation of mitotic cell cycle phase t [symbol] | 48 | 48 | 48 | 26 | 0.0112 | 0.01123 |
| GO:0006281 | DNA repair | 50 | 50 | 50 | 30 | 0.0151 | 0.01511 |

HR−

| GO.ID | Term | Annotated | Significant | Expected | Rank in classicKS | classicKS | classicFisher |
|---|---|---|---|---|---|---|---|
| GO:0000902 | cell morph [symbol] genesis | 32 | 32 | 32 | 172 | 0.472 | 1 |
| GO:0001525 | [symbol] genesis | 30 | 30 | 30 | 22 | 0.035 | 1 |
| GO:0001568 | blood vessel development | 38 | 38 | 38 | 5 | 0.011 | 1 |
| GO:0001775 | cell activation | 61 | 61 | 61 | 226 | 0. [symbol] | 1 |
| GO:0001816 | cytokine production | 36 | 36 | 36 | 302 | 0.899 | 1 |
| GO:0001817 | regulation of cytokine production | 31 | 31 | 31 | 314 | 0.95 [symbol] | 1 |
| GO:0001932 | regulation of protein phosphorylation | 47 | 47 | 47 | 158 | 0.426 | 1 |
| GO:0001934 | positive regulation of protein phosph [symbol] | 30 | 30 | 30 | 121 | 0.35 [symbol] | 1 |
| GO:0001944 | v [symbol] ure development | 38 | 38 | 38 | 6 | 0. [symbol] | 1 |
| GO:0002250 | ad [symbol] ptive [symbol] response | 30 | 30 | 30 | 237 | 0.647 | 1 |
| GO:0002252 | [symbol] effector process | 55 | 55 | 55 | 288 | 0.8 [symbol] 5 | 1 |
| GO:0002376 | [symbol] system process | 119 | 119 | 119 | 277 | 0.797 | 1 |

[symbol]

indicates data missing or illegible when filed

[symbol]

2-4. Risk Stratification Using Genes Related to Proliferate/Cell Cycle

Based on the pathway analysis, a total of 37 proliferation genes that were related to proliferation and significantly contributing to the survival results in the HR+ group were selected using the following criteria: 1) High variance, 2) Significant result in gene ontology analysis.

The 37 proliferation genes in above were analyzed to find gene prognostic predictors significantly related to DFS/DMFS (disease free survival/distant-metastasis free survival), and applied to all breast cancer subtypes to find the most significant genes related to cell proliferation through Cox proportional hazard regression analysis.

Through Cox multivariate proportional hazard regression analysis, a total of 10 genes (BUB1B, UBE2S, RRM2, KIFC1, PTTG1, MELK, CDK1, FOXMI, TRIP13, TACGAP1) were determined to have prognostic ability and independence, and these were the following for genetic risk classification. It was selected as a proliferation/cell cycle regulatory gene.

For all patient samples, the expression level of each proliferation/cell cycle regulatory gene was classified into two categories “high” or “low” according to the average expression of the gene. If the expression level of 5 or more among the 10 selected genes was classified as a low-risk group for proliferation, otherwise, a high-risk group for proliferation.

In addition to classifying patients into the molecular subtypes of breast cancer according to gene-risk stratification, patients were classified into clinical high-risk group and low-risk group based on Adjuvant! Online, as shown in Table 5.

Table. 5 showed the clinical risk assessment for each of the four molecular subtypes of breast cancer, each group being classified according to histological grade, lymph node status, and tumor size.

TABLE 5

| | | | | | | Clinical Risk |
|---|---|---|---|---|---|---|
| ER status | HER2 statue | Grade | Nodal status | Tumor Size | | In Mindact |
| ER | HER2 | well differentiated | N- | ≤3 | cm | C-low |
| positive | negative | | | 3.1-5 | cm | C-high |
| | | | 1-3 positive nodes | ≤2 | cm | C-low |
| | | | | 2.1-5 | cm | C-high |
| | | moderately differentiated | N- | ≤2 | cm | C-low |
| | | | | 2.1-5 | cm | C-high |
| | | | 1-3 positive nodes | Any size | | C-low |
| | | poorly differentiated or | N- | ≤1 | cm | C-high |
| | | undifferentiated | | 1.1-5 | cm | C-low |
| | | | 1-3 positive nodes | Any size | | C-high |
| | HER2 | well differentiated | N- | ≤2 | cm | C-low |
| | positive | OR | | 2.1-5 | cm | C-high |
| | | moderately differentiated | 1-3 positive nodes | Any size | | C-low |
| | | poorly differentiated or | N- | ≤1 | cm | C-high |
| | | undifferentiated | | 1.1-5 | cm | C-low |
| | | | 1-3 positive nodes | Any size | | C-high |
| ER | HER2 | well differentiated | N- | ≤2 | cm | C-low |
| negative | negative | | | 2.1-5 | cm | C-high |
| | | | 1-3 positive nodes | Any size | | C-low |
| | | moderately differentiated | N- | ≤1 | cm | C-high |
| | | OR | | 1.1-5 | cm | C-low |
| | | poorly differentiated or | 1-3 positive nodes | Any size | | C-high |
| | | undifferentiated | | | | |
| | HER2 | well differentiated | N- | ≤1 | cm | C-low |
| | positive | OR | | 1.1-5 | cm | C-high |

| | | | |
|---|---|---|---|
| moderately differentiated | 1-3 positive nodes | Any size | C-low |
| poorly differentiated or | Any | Any size | C-high |
| undifferentiated | | | |

[symbol]

Gene-risk stratification based on proliferation/cell cycle related genes and clinical risk evaluation criteria was subdivided into four risk groups: 1) clinically high-risk/proliferation high risk, 2) clinically high-risk/proliferation low risk, 3) clinically low-risk/proliferation high risk, and 4) clinically low-risk/proliferation low risk.

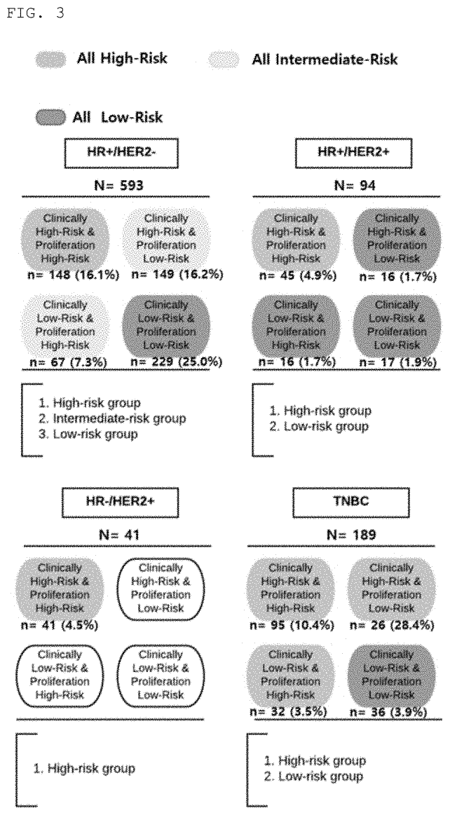

However, dividing each breast cancer subtype into the above four risk categories produced insufficient number of samples in each risk category, so each risk category within the breast cancer subtype was combined according to sample size and cox regression results (FIG. 3)

Specifically, three groups (i.e., AH, AI and AL) were generated for each subtype, clinically high-risk/proliferation high-risk group to classify All high-risk group (AH), clinically high-risk/proliferation low-risk and low-risk/proliferation high-risk to classify All intermediate group hereon, (AI), and clinically low-risk/proliferation low-risk to classify All low-risk group hereon (AL).

HR+/HER2+ subtype was divided into two risk groups that one was classified into the AH group including clinically high-risk/proliferation high-risk and the others were classified into the AL group involving the rest of them without clinically high-risk/proliferation high-risk.

All HR−/HER2+ subtype was regarded to the AH group because there was no difference between samples.

Finally, TNBC subtype was divided into two risk groups that one was classified into the AH group including clinically high-risk/proliferation high-risk, high-risk/proliferation low-risk and clinically low-risk/proliferation high-risk, the other was classified into the Al group including clinically low-risk/proliferation low-risk.

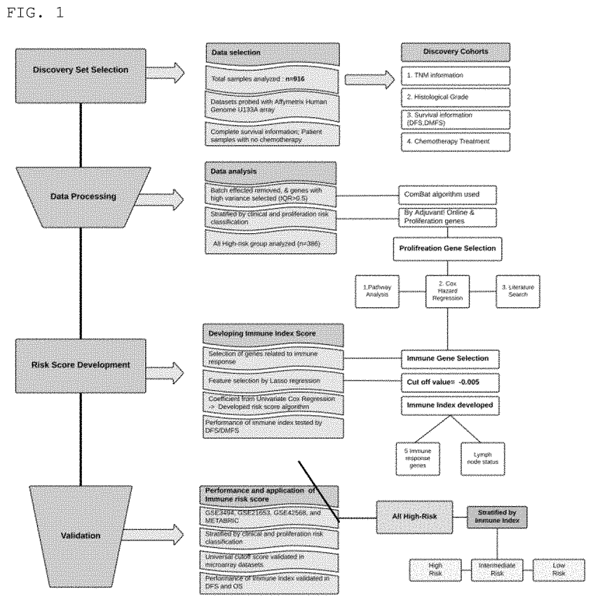

Patient samples with missing clinical information were excluded from this study. The overall schematics of this study are shown in FIG. 1. In addition, information from the validation set was also classified according to gene-risk stratification and clinical risk evaluation analyzed in a similar manner to discovery set above.

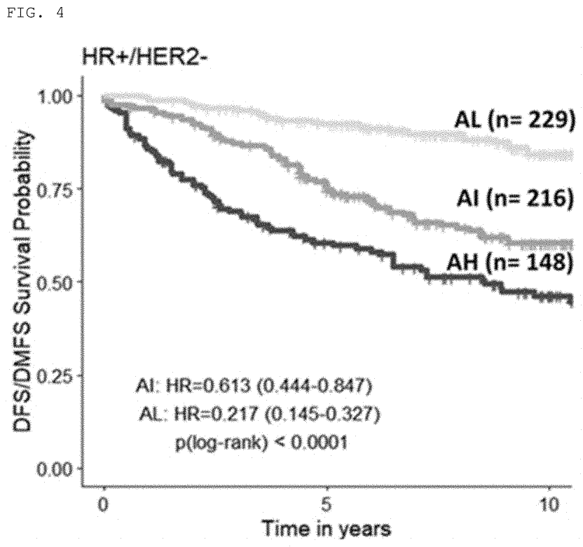

As shown in FIG. 3, survival results of the subdivided risk subgroups of breast cancer subtype using the Kaplan-Meier method and the log-rank test, also shown in FIG. 4 to FIG. 7.

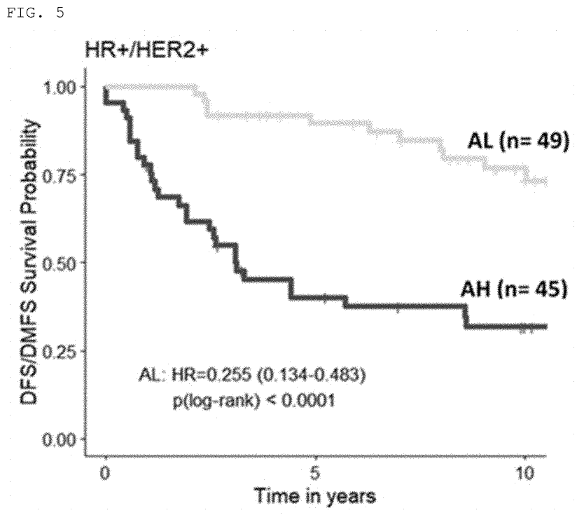

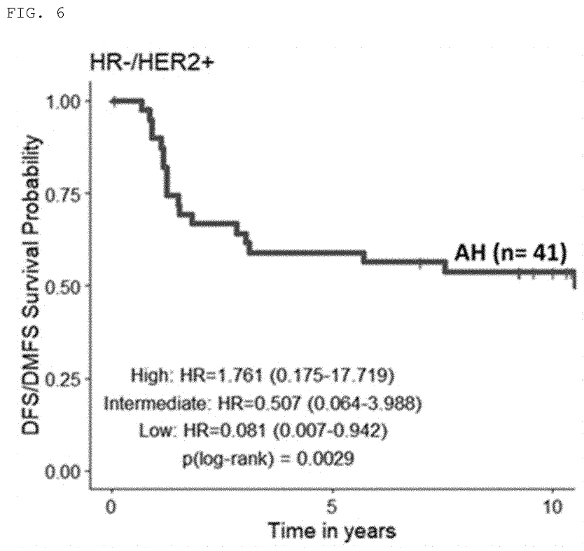

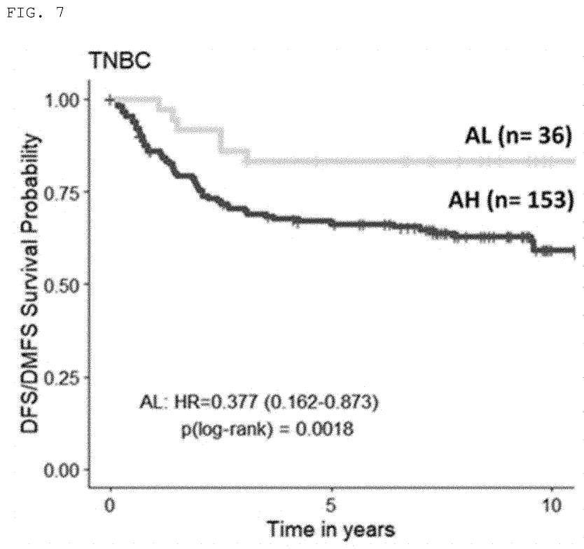

A log-rank p-values were p<0.0001 in HR+/HER2− subtype (FIG. 4) and HR+/HER2+ subtype (FIG. 5), TNBC subtype (FIG. 7) was p=0.0018. HR−/HER2+ subtype (FIG. 6) consisted of only the AH groups, so a survival curve could not be estimated. As a result of Cox regression analysis for each risk subgroup, the hazard ratio of the AI group and the AL group compared to the AH group within the HR+/HER2-subtype was 0.613 (p=0.003, 95% CI: 0.444-0.847) and 0.217 (p<0.0001, 95% CI: 0.145-0.327).

The results within the HR+/HER2+ and TNBC subtype showed similar observations, and the hazard ratio in the AL group versus the AH group were 0.255 (p<0.0001, 95% CI: 0.134-0.483) and 0.377 (p=0.0182, 95% CI: 0.162-0.873-0.08426).

### Example 3: Development of New Technology for Predicting the Prognosis of Breast Cancer Using Immune Genes Only

3-1 Primary Screening of Immune Genes Related to Predicting the Prognosis of Breast Cancer

The prognostic value of the immune response genes shown in Table 6 in each subgroup (i.e., risk group) within each breast cancer subtype was analyzed in a similar manner to that described above.

TABLE 6

| Gene code | Gene Name |
|---|---|
| TRBV20-1 | T cell receptor beta variable 20-1 |
| CCL19 | chemokine (C-C motif) ligand 19 |
| CD52 | CD52 molecule |
| SRGN | serglycin |
| CD3D | CD3d molecule, delta (CD3-TCR complex) |
| IGJ | immunoglobulin J polypeptide, linker protein for |
| | immunoglobulin alpha and mu polypeptides |
| HLA-DRA | major histocompatibility complex, class II, DR alpha |
| LOC91316 | glucuronidase, beta/immunoglobulin lambda-like |
| | polypeptide 1 pseudogene |
| IGF1 | insulin-like growth factor 1 (somatomedin C) |
| CYBRD1 | cytochrome b reductase 1 |
| TMC5 | transmembrane channel-like 5 |
| ALDH1A1 | aldehyde dehydrogenase 1 family, member A1 |
| OGN | osteoglycin |
| PDCD4 | programmed cell death 4 (neoplastic transformation |
| | inhibitor) |
| FRZB | frizzled-related protein |
| CX3CR1 | chemokine (C-X3-C motif) receptor 1 |
| IGFBP6 | insulin-like growth factor binding protein 6 |
| GLA | galactosidase, alpha |
| LOC96610 | BMS1 homolog, ribosome assembly protein (yeast) |
| | pseudogene |
| IGLL3 | immunoglobulin lambda-like polypeptide 3 |
| ITPR1 | inositol 1,4,5-triphosphate receptor, type 1 |
| SERPINA1 | serpin peptidase inhibitor, clade A (alpha-1 |
| | antiproteinase, antitrypsin), member 1 |
| EPHX2 | epoxide hydrolase 2, cytoplasmic |
| MFAP4 | microfibrillar-associated protein 4 |
| RNASET2 | ribonuclease T2 |
| CCNG1 | cyclin G1 |
| FBLN5 | fibulin 5 |
| SORBS2 | sorbin and SH3 domain containing 2 |
| CCBL2 | cysteine conjugate-beta lyase 2 |
| BTN3A2 | butyrophilin, subfamily 3, member A2 |
| TFAP2B | transcription factor AP-2 beta (activating enhancer |
| | binding protein 2 beta) |
| LTF | lactotransferrin |
| ITM2A | integral membrane protein 2A |
| HLA-DPB1 | major histocompatibility complex, class II, DP beta 1 |
| HLA-DMA | HLA-DMA major histocompatibility complex, class II, |
| | DM alpha |
| RPL3 | ribosomal protein L3 |
| LOC100130100 | similar to hCG26659 |
| FAM129A | family with sequence similarity 129, member A |
| ELOVL5 | ELOVL family member 5, elongation of long chain |
| | fatty acids (FEN1/Elo2, SUR4/Elo3-like, yeast) |
| GBP2 | guanylate binding protein 2, interferon-inducible |
| RARRES3 | retinoic acid receptor responder (tazarotene induced) 3 |
| GOLM1 | golgi membrane protein 1 |
| RTN1 | reticulon 1 |
| ICAM3 | intercellular adhesion molecule 3 |
| LAMA2 | laminin, alpha 2 |
| CXCL13 | chemokine (C-X-C motif) ligand 13 |
| ZCCHC24 | zinc finger, CCHC domain containing 24 |
| CD37 | Cluster of Differentiation 37 |
| VTCN1 | V-set domain containing T cell activation inhibitor 1 |
| PYCARD | PYD and CARD domain containing |
| CORO1A | coronin, actin binding protein, 1A |
| SH3BGRL | SH3 domain binding glutamic acid-rich protein like |
| TPSAB1 | tryptase alpha/beta 1 |
| TNFSF10 | tumor necrosis factor (ligand) superfamily, member 10 |
| ACSF2 | acyl-CoA synthetase family member 2 |
| TGFBR2 | transforming growth factor, beta receptor II (70/80 |
| | kDa) |
| DUSP4 | dual specificity phosphatase 4 |
| ARHGDIB | Rho GDP dissociation inhibitor (GDI) beta |
| TMPRSS3 | transmembrane protease, serine 3 |
| DCN | decorin |
| LRIG1 | leucine-rich repeats and immunoglobulin-like domains |
| | 1 |
| FMOD | fibromodulin |
| ZNF423 | zinc finger protein 423 |
| SQRDL | sulfide quinone reductase-like (yeast) |
| TPST2 | tyrosylprotein sulfotransferase 2 |
| CD44 | CD44 molecule (Indian blood group) |
| MREG | melanoregulin |
| GIMAP6 | GTPase, IMAP family member 6 |
| GJA1 | gap junction protein, alpha 1, 43 kDa |
| IFITM3 | interferon induced transmembrane protein 3 (1-8U) |
| BTG2 | BTG family, member 2 |
| PIP | prolactin-induced protein |
| RPS9 | ribosomal protein S9 |
| HLA-DPA1 | major histocompatibility complex, class II, DP alpha |
| | 1 |
| IMPDH2 | IMP (inosine 5′-monophosphate) dehydrogenase 2 |
| TNFRSF17 | tumor necrosis factor receptor superfamily, member 17 |
| C14orf139 | chromosome 14 open reading frame 139 |
| SPRY2 | sprouty homolog 2 (Drosophila) |
| XBP1 | X-box binding protein 1 |
| THYN1 | thymocyte nuclear protein 1 |
| APOD | apolipoprotein D |
| C10orf116 | chromosome 10 open reading frame 116 |
| VAV3 | vav 3 guanine nucleotide exchange factor |
| FAS | Fas (TNF receptor superfamily, member 6) |
| MYBPC1 | myosin binding protein C, slow type |
| CFB | complement factor B |
| TRIM22 | tripartite motif-containing 22 |
| ARID5B | AT rich interactive domain 5B (MRF1-like) |
| PTGDS | prostaglandin D2 synthase 21 kDa (brain) |
| TGFBR3 | transforming growth factor, beta receptor III |
| TNFAIP8 | tumor necrosis factor, alpha-induced protein 8 |
| SEMA3C | sema domain, immunoglobulin domain (Ig), short |
| | basic domain, secreted, (semaphorin) 3C |
| TMEM135 | transmembrane protein 135 |
| ARHGEF3 | Rho guanine nucleotide exchange factor (GEF) 3 |
| PTGER4 | prostaglandin E receptor 4 (subtype EP4) |
| ABCA8 | ATP-binding cassette, sub-family A (ABC1), member |
| | 8 |
| ICAM2 | intercellular adhesion molecule 2 |
| HLA-DQB1 | major histocompatibility complex, class II, DQ beta 1 |
| HSPA2 | heat shock 70 kDa protein 2 |
| CD27 | CD27 molecule |
| ARMCX1 | armadillo repeat containing, X-linked 1 |
| POU2AF1 | POU class 2 associating factor 1 |
| IGBP1 | immunoglobulin (CD79A) binding protein 1 |
| PDE4B | phosphodiesterase 4B, CAMP-specific |
| ADH1B | alcohol dehydrogenase 1B (class I), beta polypeptide |
| WLS | wntless homolog (Drosophila) |
| SUCLG2 | succinate-CoA ligase, GDP-forming, beta subunit |
| PGR | progesterone receptor |
| STARD13 | StAR-related lipid transfer (START) domain |
| | containing 13 |
| SORL1 | sortilin-related receptor, L(DLR class) A |
| | repeats-containing |
| ATP1B1 | ATPase, Na+/K+ transporting, beta 1 polypeptide |
| IFT46 | intraflagellar transport 46 homolog (Chlamydomonas) |
| SIK3 | SIK family kinase 3 |
| LIPT1 | lipoyltransferase 1 |
| OMD | osteomodulin |
| HBB | hemoglobin, beta |
| C3 | complement component 3 |
| FGL2 | fibrinogen-like 2 |
| PECI | peroxisomal D3,D2-enoyl-CoA isomerase |
| RAC2 | ras-related C3 botulinum toxin substrate 2 (rho family, |
| | small GTP binding protein Rac2) |
| PDZRN3 | PDZ domain containing ring finger 3 |
| CXCL12 | chemokine (C-X-C motif) ligand 12 |
| DPYD | dihydropyrimidine dehydrogenase |
| TXNDC15 | thioredoxin domain containing 15 |
| STOM | stomatin |
| EMCN | endomucin |
| SCGB2A2 | secretoglobin, family 2A, member 2 |
| FAM176B | family with sequence similarity 176, member B |
| HIGD1A | HIG1 hypoxia inducible domain family, member 1A |
| ACSL5 | acyl-CoA synthetase long-chain family member 5 |
| RPS24 | ribosomal protein S24 |
| RGS10 | regulator of G-protein signaling 10 |
| RAI2 | retinoic acid induced 2 |
| CNN3 | calponin 3, acidic |
| FBXW4 | F-box and WD repeat domain containing 4 |
| SEPP1 | selenoprotein P, plasma, 1 |
| SLC44A4 | solute carrier family 44, member 4 |
| MGP | matrix Gla protein |
| ABCD3 | ATP-binding cassette, sub-family D (ALD), member 3 |
| SETBP1 | SET binding protein 1 |
| APOBEC3G | apolipoprotein B mRNA editing enzyme, catalytic |
| | polypeptide-like 3G |
| LCP2 | lymphocyte cytosolic protein 2 (SH2 domain |
| | containing leukocyte protein of 76 kDa) |
| HLA-DRB1 | major histocompatibility complex, class II, DR beta 1 |
| SCUBE2 | signal peptide, CUB domain, EGF-like 2 |
| DEPDC6 | DEP domain containing 6 |
| RPL15 | ribosomal protein L15 |
| SH3BP4 | SH3-domain binding protein 4 |
| MSX2 | msh homeobox 2 |
| CLU | clusterin |
| DPT | dermatopontin |
| ZNF238 | zinc finger protein 238 |
| HBP1 | HMG-box transcription factor 1 |
| GSTK1 | glutathione S-transferase kappa 1 |
| ZBTB16 | zinc finger and BTB domain containing 16 |
| CCDC69 | coiled-coil domain containing 69 |
| ALDH2 | aldehyde dehydrogenase 2 family (mitochondrial) |
| SLC1A1 | solute carrier family 1 (neuronal/epithelial high |
| | affinity glutamate transporter, system Xag), member 1 |
| ARMCX2 | armadillo repeat containing, X-linked 2 |
| HMGCS2 | 3-hydroxy-3-methylglutaryl-CoA synthase 2 |
| | (mitochondrial) |
| TSPAN3 | tetraspanin 3 |
| FTO | fat mass and obesity associated |
| PON2 | paraoxonase 2 |
| C16orf62 | chromosome 16 open reading frame 62 |
| QDPR | quinoid dihydropteridine reductase |
| LRP2 | low density lipoprotein receptor-related protein 2 |
| PSMB8 | proteasome (prosome, macropain) subunit, beta type, 8 |
| | (large multifunctional peptidase 7) |
| HCLS1 | hematopoietic cell-specific Lyn substrate 1 |
| FXYD1 | FXYD domain containing ion transport regulator 1 |
| OAT | ornithine aminotransferase |
| SLC38A1 | solute carrier family 38, member 1 |
| MAOA | monoamine oxidase A |
| LPL | lipoprotein lipase |
| C10orf57 | chromosome 10 open reading frame 57 |
| SPARCL1 | SPARC-like 1 (hevin) |
| ERAP2 | endoplasmic reticulum aminopeptidase 2 |
| PDGFRL | platelet-derived growth factor receptor-like |
| RBP4 | retinol binding protein 4, plasma |
| LRRC17 | leucine rich repeat containing 17 |
| LHFP | lipoma HMGIC fusion partner |
| BLNK | B-cell linker |
| HBA2 | hemoglobin, alpha 2 |
| CST7 | cystatin F (leukocystatin) |
| TRAT1 | T-cell receptor-associated transmembrane adapter 1 |
| IL21R | Interleukin-21 receptor |
| IGHM | Immunoglobulin heavy constant mu |
| CTLA4 | Cytotoxic T-lymphocyte protein 4 |
| IL2RB | Interleukin-2 receptor subunit beta |
| TNFRSF9 | Tumor necrosis factor receptor superfamily member 9 |
| CTSW | Cathepsin W |
| CCR10 | C-C chemokine receptor type 10 |
| GPR18 | G Protein-Coupled Receptor 18 |
| CR2 | Complement receptor type 2 |
| DOCK10 | Dedicator Of Cytokinesis 10 |
| GZMB | Granzyme B |
| ITK | IL2 Inducible T Cell Kinase |
| LTB | Lymphotoxin Beta |
| IGLJ3 | Immunoglobulin lambda joining 3 |
| IGLV1-44 | Immunoglobulin lambda variable 1-44 |
| AIM2 | Absent In Melanoma 2 |
| CXCL9 | C-X-C motif chemokine 9 |
| KIAA0125 | long non-conding RNA |
| IL2RG | Interleukin 2 Receptor Subunit Gamma |
| CD69 | Cluster of Differentiation 69 |
| CD55 | Cluster of Differentiation 55 |
| TRAF3IP3 | TRAF3 Interacting Protein 3 |
| EVI2B | Ecotropic Viral Integration Site 2B |
| STAP1 | Signal-transducing adaptor protein 1 |
| KLRB1 | Killer cell lectin-like receptor subfamily B member 1 |
| PRKCB | Protein kinase C beta type |
| GPR171 | G Protein-Coupled Receptor 171 |
| PPP1R16B | Protein phosphatase 1 regulatory inhibitor subunit 16B |
| SH2D1A | SH2 domain-containing protein 1A |
| TNFRSF1B | Tumor necrosis factor receptor superfamily member |
| | 1B |
| CD48 | Cluster of Differentiation 48 |
| BANK1 | B-cell scaffold protein with ankyrin repeats 1 |
| LY9 | T-lymphocyte surface antigen Ly-9 |

A total of 110 immune genes were primary selected in their relevance to MHC-1, MHC-2, T-cells, and B-cells and their relevance to the immune response. Cox regression univariate analysis was performed on the 110 immune response genes, and their significance was observed for each breast cancer molecular subtype. Cox regression analysis was performed in the same manner as in Example 2-2 above. Table 7 showed the 10 most significant immune response genes for each breast cancer subtype.

In each breast cancer subtype, all AH groups had increased expression of immune response genes significantly related to the positive prognosis. As a result of univariate analysis, in the HR+/HER2− subtype group, 55 immune response genes showed significant p-values (p<0.05), and all had negative coefficient values, and also showed a positive correlation with prolonged survival. In a similar manner, all high-risk groups within HR+/HER2+, HR−/HER2+ and TNBC subtype possessed 96, 30 and 8 immune response genes, respectively, showing significant p-value (p<0.05), which were all had negative coefficients.

In contrast to the observation in the AH group, the effect of immune response genes was less pronounced in the Al and AL groups. HR+/HER2− subtype in the AI group had no significant survival-related immune response gene and the lowest p-value among all genes was 0.09 or higher. The AL group had several significant genes, but their hazard ratio was not associated with positive DFS/DMFS results.

Based on the results of Cox regression analysis, it focused to AH group to further investigate genes with prognostic predictive ability.

TABLE 7

| | coef | hr | se(coef) | z | pvalue |
|---|---|---|---|---|---|
| HR+/HER2− | | | | | |
| CD69 | −0.68986 | 0.501646 | 0.211284 | −3.26509 | 0.001094 |
| CD55 | −1.02617 | 0.358375 | 0.323082 | −3.1762 | 0.001492 |
| TRAF3IP3 | −0.93552 | 0.392382 | 0.311055 | −3.00757 | 0.002633 |
| EVI2B | −0.91907 | 0.398891 | 0.30849 | −2.97923 | 0.00289 |
| IL21R | −0.80724 | 0.44609 | 0.273252 | −2.95418 | 0.003135 |
| IGHM | −0.42671 | 0.652655 | 0.148793 | −2.86779 | 0.004134 |
| IGJ | −0.38369 | 0.681341 | 0.135338 | −2.83506 | 0.004582 |
| CR2 | −0.46309 | 0.629334 | 0.164106 | −2.82192 | 0.004774 |
| GZMB | −0.553 | 0.575221 | 0.20029 | −2.761 | 0.005762 |
| STAP1 | −0.59319 | 0.552562 | 0.218993 | −2.70871 | 0.006755 |
| HR+/HER2+ | | | | | |
| KLRB1 | −1.52862 | 0.216835 | 0.325476 | −4.69656 | 2.65E−06 |
| PRKCB | −2.19827 | 0.110995 | 0.468355 | −4.6936 | 2.68E−06 |
| CD37 | −1.93956 | 0.143767 | 0.413562 | −4.68989 | 2.73E−06 |
| GPR171 | −2.04521 | 0.129353 | 0.436535 | −4.6851 | 2.80E−06 |
| CD3D | −1.92875 | 0.14533 | 0.417865 | −4.61572 | 3.92E−06 |
| PPP1R16B | −1.67262 | 0.187754 | 0.365964 | −4.57047 | 4.87E−06 |
| ITK | −2.57119 | 0.076444 | 0.564613 | −4.5539 | 5.27E−06 |
| SH2D1A | −2.41604 | 0.089274 | 0.531415 | −4.54643 | 5.46E−06 |
| TNFRSF1B | −3.84986 | 0.021283 | 0.847878 | −4.54058 | 5.61E−06 |
| CD48 | −2.60977 | 0.073551 | 0.582633 | −4.47927 | 7.49E−06 |
| HR−/HER2+ | | | | | |
| BANK1 | −0.94469 | 0.3888 | 0.261254 | −3.61599 | 0.000299 |
| GIMAP6 | −2.0162 | 0.13316 | 0.644425 | −3.12868 | 0.001756 |
| CD69 | −1.61401 | 0.199087 | 0.542267 | −2.97642 | 0.002916 |
| GPR18 | −0.94085 | 0.390294 | 0.335457 | −2.80469 | 0.005036 |
| LY9 | −0.99188 | 0.370877 | 0.353694 | −2.80436 | 0.005042 |
| VNN2 | −1.05607 | 0.347819 | 0.388099 | −2.72115 | 0.006506 |
| TCL1A | −2.0106 | 0.133909 | 0.742011 | −2.70966 | 0.006735 |
| CYTIP | −1.61108 | 0.199672 | 0.606694 | −2.6555 | 0.007919 |
| CTSW | −0.89989 | 0.406614 | 0.351074 | −2.56325 | 0.01037 |
| PTPRC | −1.2287 | 0.292673 | 0.483899 | −2.53916 | 0.011112 |
| TNBC | | | | | |
| PDCDILG2 | −0.67776 | 0.507751 | 0.258882 | −2.61804 | 0.008844 |
| LTA | −1.2035 | 0.300141 | 0.484417 | −2.48444 | 0.012976 |
| IGLV1-44 | −0.39441 | 0.674078 | 0.1651 | −2.38891 | 0.016898 |
| CCR10 | −0.75399 | 0.470485 | 0.329995 | −2.28486 | 0.022321 |
| TNFRSF9 | −0.8434 | 0.430247 | 0.370424 | −2.27684 | 0.022796 |
| GPR18 | −0.47547 | 0.621591 | 0.218 | −2.18107 | 0.029178 |
| IGLJ3 | −0.49076 | 0.612164 | 0.232956 | −2.10664 | 0.035148 |
| IGHG1 | −0.54498 | 0.579854 | 0.268435 | −2.03021 | 0.042335 |
| IGHM | −0.29906 | 0.741512 | 0.153638 | −1.94655 | 0.051589 |
| CD19 | −0.49133 | 0.611815 | 0.254689 | −1.92912 | 0.053716 |

3-2. Screening and Selection of Main Immune Response Genes

Using a Lasso regression analysis, we tried to further selected significant immune response genes related to patient survival in each breast cancer subtype. First, Lasso feature selection method was used to select the most significant immune response genes in relation to DFS/DMFS, and applied to ‘coxnet’ of R version 3.4.3. to find the optimal lambda value by 10,000 fold cross-validation. And then the active covariate was found by the Lasso method. As mentioned above, the most significant genes were selected by performing Lasso regression analysis, and these results were verified through Cox proportional hazard univariate analysis.

Further details, the AH group was integrated in each breast cancer subtype. Patient data with missing or ambiguous clinical information were excluded from this analysis and subsequently analyzed during the development of prognostic models. 9 active genes (CTLA4, CTSW, DOCK10, GPR18, IGHM, IL21R, IL2RB, TNFRSF9, and TRAT1) that negatively affect hazard were selected by Lasso regression analysis, and were shown in Table 8 below. In addition, 5 genes (TRAT1, IGHM, IL21R, GZMB, GPR18) by Cox regression analysis that had a significant effect (p<0.0001) on hazard was discovered, and the analysis results of these genes were shown in Table 9 below.

Finally, 5 genes (TRAT1, IL21R, IGHM, CTLA4, IL2RB) with negative coefficient value of less than −0.05 were selected (See Table 8).

TABLE 8

| Gene | Coefficient |
|---|---|
| TRAT1 | −0.13118865 |
| IL21R | −0.10504567 |
| IGHM | −0.0997505 |
| CTLA4 | −0.09963025 |
| IL2RB | −0.08664438 |
| INFRSF9 | −0.04891361 |
| CTSW | −0.04188042 |
| CCR10 | −0.01014675 |
| GPR18 | −0.00497377 |
| CR2 | −0.00196256 |
| DOCK10 | 0.240217798 |

TABLE 9

| | coef | hr | se(coef) | z | pvalue |
|---|---|---|---|---|---|
| TRAT1 | −0.38483917 | 0.68056 | 0.090824 | −4.23721 | 2.26E−05 |
| IGHM | −0.36913285 | 0.691334 | 0.08911 | −4.14242 | 3.44E−05 |
| IL21R | −0.63111572 | 0.531998 | 0.155136 | −4.06814 | 4.74E−05 |
| GZMB | −0.43362517 | 0.648155 | 0.109901 | −3.94561 | 7.96E−05 |
| GPR18 | −0.51640599 | 0.596661 | 0.132572 | −3.89528 | 9.81E−05 |
| CTSW | −0.424037 | 0.6544 | 0.110839 | −3.82572 | 0.00013 |
| EVI2B | −0.69469314 | 0.499228 | 0.184028 | −3.77492 | 0.00016 |
| CORO1A | −0.65641984 | 0.518705 | 0.174807 | −3.75512 | 0.000173 |
| CTLA4 | −0.50054211 | 0.606202 | 0.133582 | −3.74706 | 0.000179 |
| ITK | −0.61326229 | 0.541581 | 0.163868 | −3.74242 | 0.000182 |
| LTB | −0.50805218 | 0.601666 | 0.138251 | −3.67486 | 0.000238 |
| IGLJ3 | −0.5058777 | 0.602976 | 0.137725 | −3.67311 | 0.00024 |
| IGLV1-44 | −0.36467637 | 0.694421 | 0.099401 | −3.66876 | 0.000244 |
| AIM2 | −0.70455447 | 0.494329 | 0.192936 | −3.65175 | 0.00026 |
| CXCL9 | −0.32522115 | 0.722368 | 0.091129 | −3.56895 | 0.000358 |
| IL2RB | −0.76753583 | 0.464155 | 0.216092 | −3.5519 | 0.000382 |
| CXCL13 | −0.23298293 | 0.792167 | 0.065837 | −3.53879 | 0.000402 |
| KIAA0125 | −0.8382797 | 0.432454 | 0.237175 | −3.53444 | 0.000409 |
| IL2RG | −0.58234889 | 0.558585 | 0.165644 | −3.51567 | 0.000439 |

3-3. Production of a Risk Score Calculation Model for Predicting the Prognosis of Early Breast Cancer

A model for predicting the prognosis of breast cancer was created by combining the five immune genes selected in Example 3-2. The inventors of the present invention have confirmed that a breast cancer prognosis risk score could be calculated by performing a linear combination of the expression value of each of the five immune genes selected above and Cox Regression estimates (used as coefficients). The Cox regression estimate of each gene is shown in Table 10 below.

TABLE 10

| | Cox Regression estimate | Cox Regression |
|---|---|---|
| Gene | 95% confidence interval | point estimate |
| TRAT1 | −0.567144, −0.1952896 | −0.3812 |
| IL21R | −0.9759746, −0.3412672 | −0.6586 |
| CTLA4 | −0.7454524, −0.2010003 | −0.4732 |
| IGHM | −0.5428339, −0.1855019 | −0.3642 |
| IL2RB | −1.146983, −0.266771 | −0.7069 |
| Lymph node | 0.3910642, 1.013551 | 0.7023 * 2 |
| infiltration | | |
| status | | |

In particular, in order to include information on clinical variables for more accurate prediction, Cox univariate and multivariate analysis were performed on the clinical variables, as a result, among the clinical variables, it was confirmed that the breast cancer infiltration status in the lymph nodes (herein, abbreviated as ‘lymph nodes status’) had the most significant effect on survival as an independent prognostic factor (data not shown). Accordingly, a risk score calculation formula for predicting breast cancer prognosis was produced as follows using the Cox regression estimate for the lymph node state. As described below, the risk score calculated by the present invention was genetic information and was also referred to as an ‘immune index’ in the present specification because it included only immune genes.

risk score={(−0.3812*χ_{TRAT1})+(−0.6586*χ_{IL21R})+(−0.3642*χ_{IGHM})+(−0.4732*χ_{CTLA4})+(−0.7069*χ_{IL2RB})}+(0.7023*2*LN) [Formula 3]

In Formula 3, x is the expression value of a gene indicated by a subscript, and N is an integer indicating the presence of LN.

### Example 4: Confirmation of Prognostic Performance of Breast Cancer Prognostic Model of the Present Invention Using Immune Response Genes

In Example 3-3 above, the risk index of each patient in the discovery set was calculated according to the risk index calculation formula prepared. Based on the risk index (immunity index), patient samples within the AH group were further stratified into specific risk groups. In the present invention, the performance of the risk score was tested in two parts: 1) Hazard index as a continuous variable, 2) Risk index based on the optimal cutoff point derived using rank statistics, maximally selected from the R version 3.4.3.′ survminer′ package by the bootstrapping method.

We hypothesized that a lower (more negative) risk index was associated with a reduced chance of recurrence as well as prolonged survival.

Table 11 below showed the results of univariate analysis and multivariate analysis performed in relation to the risk index of the present invention, respectively. Continuous risk index based on univariate analysis was significantly and highly associated with relapse result (p<0.0001, Table 11).

Statistical significance was also confirmed in multivariate analysis of risk index and clinical factors, and as the risk index increased, the risk index was the most prominent variable associated with recurrence, and the hazard ratio of 1.46 (p<0.0001, 95% CI: 1.30-1.65) appeared (Table 11). These results suggested that a lower risk score is associated with a reduced chance of recurrence as well as long-term survival.

TABLE 11

| | Hazard Ratio | 95% CI | P value |
|---|---|---|---|
| | Univariate Analysis | | |
| Number of patients n = 386 | | | |
| Event = 181 | | | |
| Risk Score: Risk Optimal | | | |
| High | 1.00 | | |
| Intermediate | 0.42 | 0.29-0.60 | <0.0001 |
| Low | 0.17 | 0.10-0.29 | <0.0001 |
| Clinical Variables: | | | |
| Lymph node infiltration | | | |
| 0 | 1.00 | | |
| 1 | 2.02 | 1.48-2.76 | <0.0001 |
| Histological grade | | | |
| High | 1.00 | | |
| Low&Intermediate | 1.24 | 0.93-1.67 | 0.146 |
| Tumor size | | | |
| A | 1.00 | | |
| B | 0.75 | 0.55-1.02 | 0.0679 |
| Age | | | |
| A | 1.00 | | |
| B | 1.10 | 0.81-1.48 | 0.55 |
| | Multivariate Analysis | | |
| Number of patients n = 386 | | | |
| Event = 181 | | | |
| Risk Score: Risk Optimal | | | |
| High | 1.00 | | |
| Intermediate | 0.49 | 0.32-0.73 | 0.0004 |
| Low | 0.21 | 0.12-0.37 | <0.0001 |
| Clinical Variables: | | | |
| Lymph node infiltration | | | |
| 0 | 1.00 | | |
| 1 | 1.31 | 0914-1.88 | 0.14044 |

Two optimal points were selected through bootstrapping of the maximally selected rank statistics. The optimal cutoff point of the risk index according to the model of the present invention was obtained by bootstrapping the most selected statistics in the ‘survminer’ package (R version 3.4.3.).

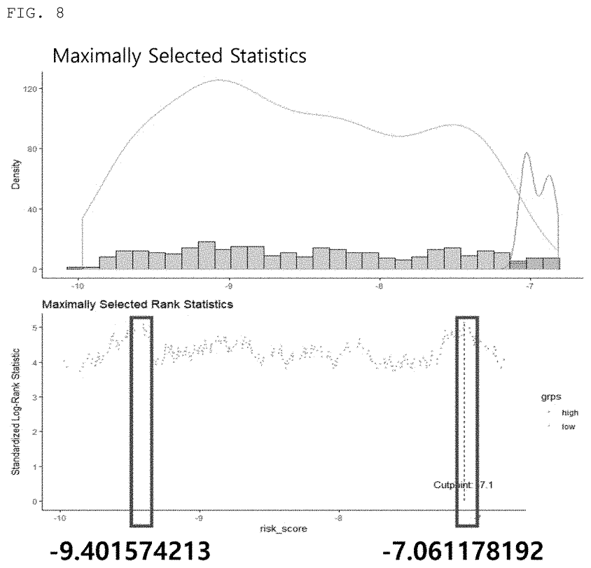

FIG. 8 shows two cutoff points identified by the bootstrapping method. When the risk index of patients is normalized by the bootstrapping method and expressed as a distribution, based on the reliability of 85%, the cut-off value for the 2.5 percentile was set as cutoff-1, and 97.5 the cutoff value for the quartile was cutoff-2. As a result, the two cutoff values calculated by the bootstrapping method were −9.4 and −7.1, and if it was less than −9.4, it was classified as low-risk, if it was between −9.4 and −7.1, it was classified as intermediate risk, and −7.1 if it is greater than that, it is classified as high-risk.

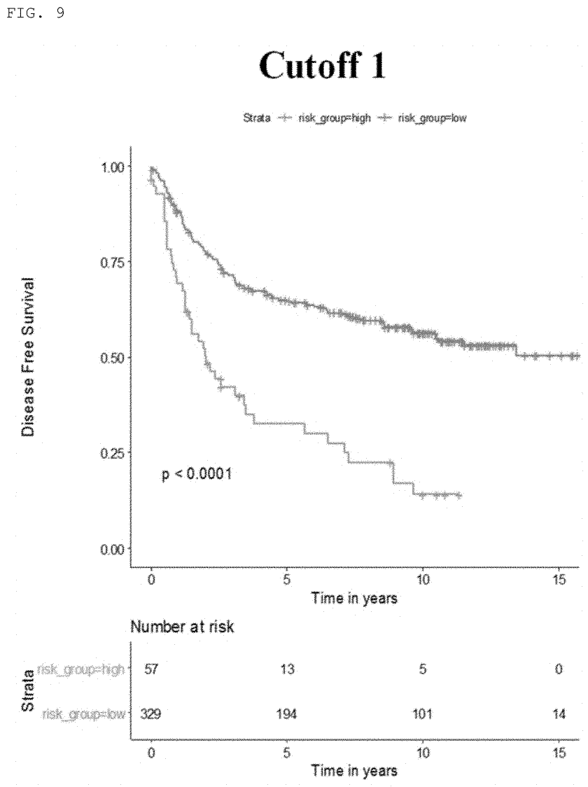

Cutoff-2 (−9.401574213, rounded to −9.4) stratified the low-risk group and the high-risk group, and the low-risk group had a hazard ratio of 0.35 (p=0.0001, 95% CI: 0.25-0.50) (FIG. 9).

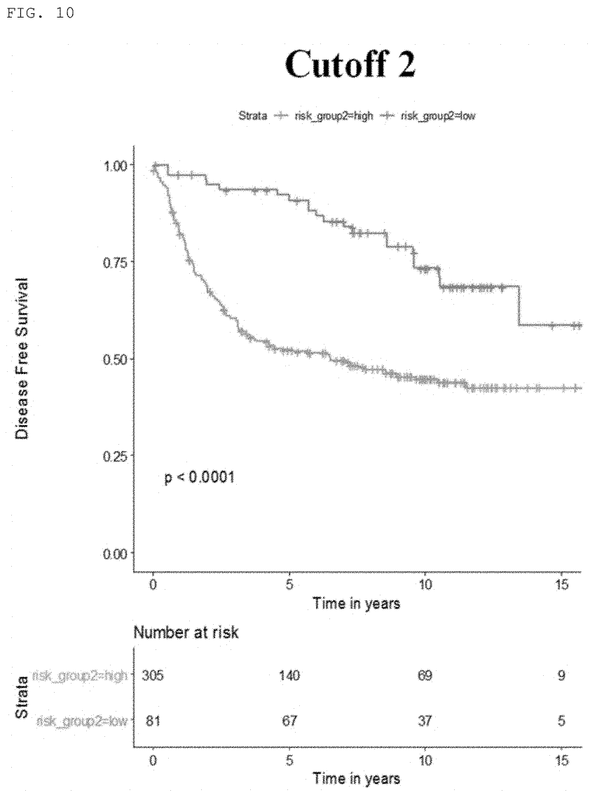

The group stratified by cutoff −1 (−7.061178192, rounded to −7.1) revealed a significant difference in recurrence rate, and a hazard ratio of 0.35 (p<0.0001, CI: 0.22-0.56) (FIG. 10).

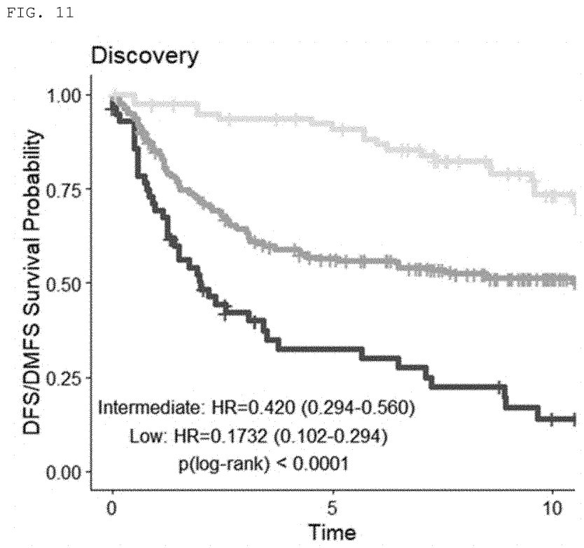

Two optimal cutoff points (i.e., cutoff-1 and cutoff-2) were used together, and those classified differently as high or low risk according to the two cutoff points were classified as an intermediate group (Table 11 and FIG. 8). By applying this to the risk score of the present invention, three risk groups were created based on the risk index: immune high-risk, immune intermediate-risk, and immune low-risk (Table 11 and FIG. 11). FIG. 11 shows survival curves of a discovery set stratified into three risk groups. All three risk groups showed statistically significant differences. Compared to the high-risk group, the hazard ratio of the intermediate-risk group was 0.42 (p<0.0001, CI: 0.29-0.56), and the hazard ratio of the low-risk group was 0.17 (p<0.0001, CI: 0.10-0.29).

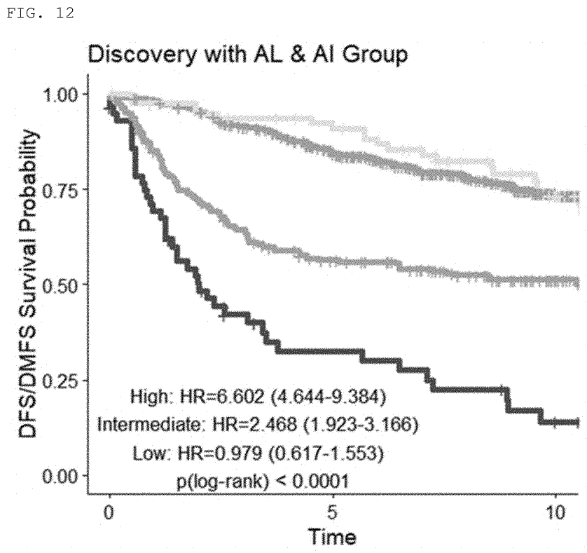

5-year survival rate was 90.9% in the low-risk group, 56.4% in the low-risk group, 32.5% in the high-risk group. In addition, 10-year survival rate decreased to 73.4% in the low-risk group, 51.3% in the intermediate-risk group, and 14.1% in the low-risk group. FIG. 12 is basically the same as FIG. 11, but showed the survival curves for the AL group and the AI group, which were excluded in the development of the prognostic prediction model (formular) of the present invention. As shown in FIG. 12, there was no statistical difference between the immune low-risk group and the AL&AI group classified according to the judgment using the risk index of the present invention, while comparing the immune intermediate-risk group and the immune low-risk group it showed that a statistically significant difference appears.

In order to find out whether the risk index according to the invention has independence for the prediction of breast cancer recurrence, the risk index was verified through multivariate analysis, which is shown in Table 11 above.

When adjusted for conventional clinicopathological parameters, the risk index (immunological marker) showed statistical significance by multivariate analysis.

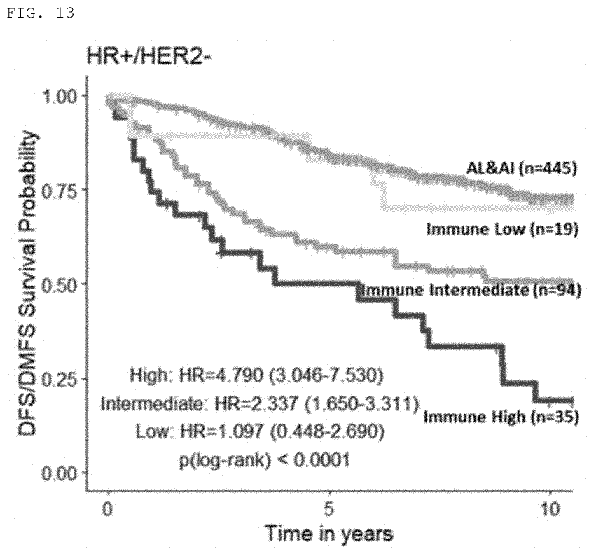

In addition, each molecular subtype of breast cancer (HR+/HER2−, HR+/HER2+, HR−/HER2+, TNBC) was tested by applying the risk index model of the present invention. The survival curves of the intermediate-risk group and the immune-low risk group are shown in FIGS. 13 to 16.

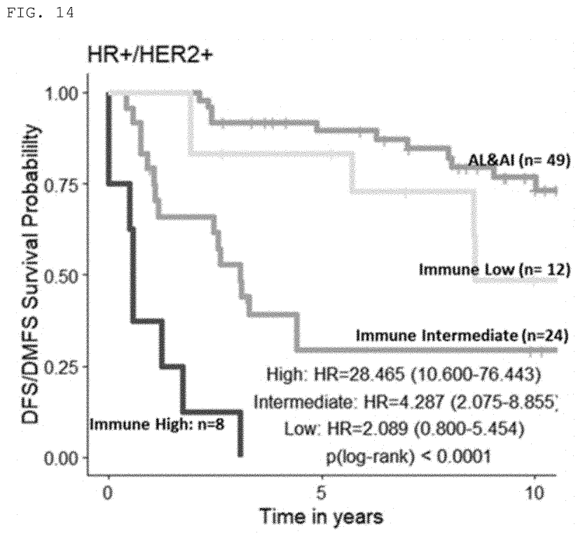

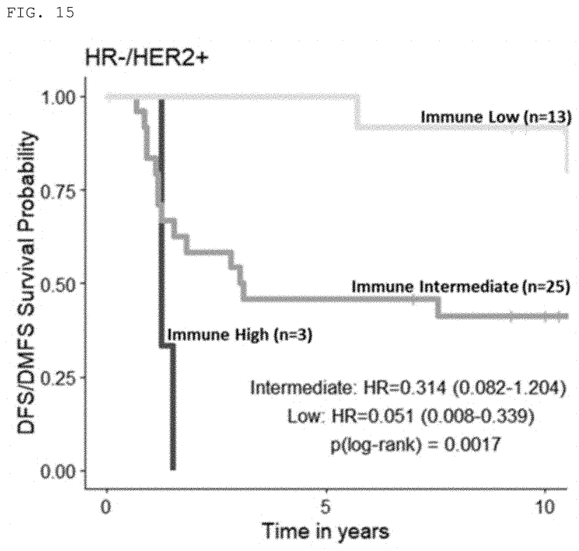

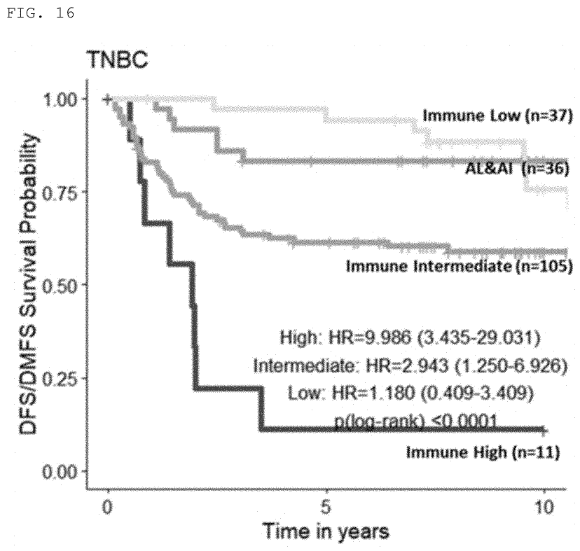

Excluding FIG. 15 (since all HR−/HER2+ subtype groups were classified as AH, see examples 2-4 above), FIG. 13(HR+/HER2−), FIG. 14(HR+/HER2+), and FIG. 16(TNBC), together with the survival curves for the AL & AI groups, the survival curve of the AL & AI group showed a tendency similar to that of the immune low-risk group according to the risk index of the present invention. As shown in FIGS. 13 to 16, the risk index (immune index) of the present invention was statistically significant in all four molecular subtypes of breast cancer (p<0.05).

### Example 5: Verification of Prognostic Performance of Breast Caner Prognostic Model of the Present Invention Using Immune Genes

Unlike the discovery set used in above embodiments, in order to expand the scope of application of the breast cancer prognosis prediction model (risk index calculation model) of the present invention, cohorts on various other platforms are used in the present invention: The breast cancer prognostic risk index model of the present invention was tested by a total of three different test set (i.e. validation set): two different microarray platform sets and another validation set using METABRIC data. As a microarray platform set, GSE3494, which was selected as the first validation set, was the same platform as the cohort of the discovery set (Affymetrix GPL96). The second validation set consisted of two cohorts, GSE21653 and GSE42568 (Affymetrix GPL570).

In order to stratify patients within the AH group into immune low-risk groups and immune high-risk groups by applying the risk index of the present invention, the optimal cutoff value −7.1 (cutoff-1, see Example 4 above) was applied to the validation set. In the validation set, there was no sample showing a risk index as low as −9.4 (cutoff-2, see Example 4 above), which was thought to be due to the difference in whether patients performed chemotherapy between the discovery set and the validation set.

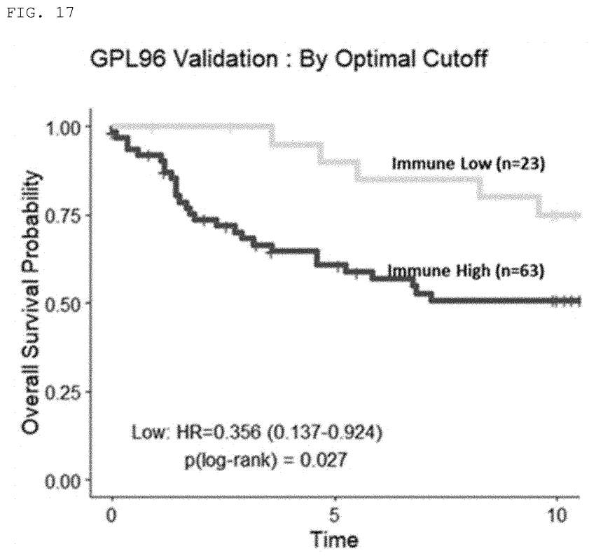

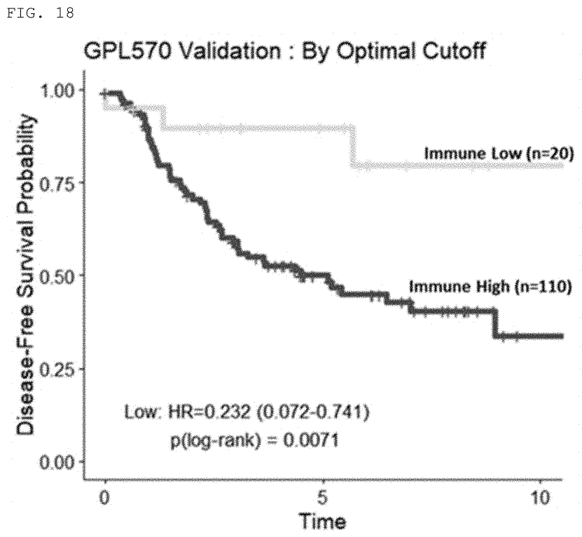

FIGS. 17, 18, and 19 showed survival curves in three validation sets of Affymetrix GPL96, Affymetrix GPL570, and METABRIC, respectively, and showed that there was a significant difference in recurrence and survival between the low-risk group and the high-risk group. FIG. 17 showed the OS (overall survival) difference between the immune high-risk group and the immune low-risk group and the immune low-risk group defined by the risk index (immunity index) of the present invention in the GSE3494 cohort, wherein in hazard ratio in the immune low-risk group was 0.36 (p=0.0339, CI: 0.14-0.92). FIG. 18 showed the possibility of recurrence between the immune high-risk group and the immune low-risk group defined by the risk index of the present invention in the validation set consisting of GSE21653 and GSE42568, wherein the hazard ratio in the immune low-risk group was 0.24 (p=0.0137, CI: 0.07-0.74). In both validation sets, the risk index (immunity index) successfully classified the immune low-risk group and the immune high-risk group, and showed a statistical difference in survival results.

In addition, in the first validation set (GSE3494), the 5-year overall survival rates of the low-risk group and high-risk group were 90.0% and 60.9%, respectively. In the second validation set (GSE42683 and GSE21653), the low-risk group and the year-DFS of the high-risk group was 89.7% and 50.0%, respectively. In the first validation set, the 10-year overall survival rates of the low-risk and high-risk groups were 75.0% and 50.8%, respectively. In second validation set, the recurrence rates of the low-risk and high-risk groups were 798 and 33.7%, respectively. In addition, as a result of performing univariate analysis and multivariate analysis on each validation set, the risk index of the present invention was found to be the largest variable in predicting prognosis after adjustment (Tables 12 and 13). Taken together, based on the results from the microarray validation sets, the risk model for predicting breast cancer prognosis of the present invention demonstrated robustness (robustness or robustness) in predicting overall survival (OS) and recurrence (p<0.05).

TABLE 12

| | Hazard Ratio | 95% CI | F value |
|---|---|---|---|
| | Univariate Analysis (GSE3494) | | |
| Number of patients n = 86 | | | |
| Events = 33 | | | |
| Risk Score: | | | |
| Continous | | | |
| As score increases | 2.24 | 1.41-3.37 | 0.000664 |
| Risk Optimal | | | |
| High | 1.00 | | |
| Low | 0.36 | 0.14-0.92 | 0.0339 |
| Clinical Variables: | | | |
| Lymph node infiltration | | | |
| 0 | 1.00 | | |
| 1 | 2.74 | 1.30-5.80 | 0.00824 |
| Histological grade | | | |
| High | 1.00 | | |
| Low&Intermediate | 1.03 | 0.52-2.04 | 0.937 |
| Tumor size | | | |
| A | 1.00 | | |
| B | 2.36 | 0.83-6.73 | 0.107 |
| Age | | | |
| A | 1.00 | | |
| B | 1.39 | 0.66-2.93 | 0.382 |
| | Multivariate Analysis (GSE3494) | | |
| Number of patients n = 86 | | | |
| Events = 33 | | | |
| Risk Score: | | | |
| Continous | | | |
| As score increases | 2.73 | 1.13-6.58 | 0.0252 |
| Clinical Variables: | | | |
| Lymph node infiltration | | | |
| 0 | 1.00 | | |
| 1 | 0.68 | 0.17-2.88 | 0.604 |

The classification in the table is based on the classification of clinical variables commonly used in breast cancer (AGE: 50 or greater=A otherwise B (50<=A 50>B; Size: B>2 cm otherwise A; Histological grade: →1: low, 2: intermediate, 3: high).

TABLE 13

| | Univariate Analysis (GSE42563 & GSB21563) | | |
|---|---|---|---|
| Number of patients | | | |
| n = 130 | | | |
| Risk Score: | | | |
| Continous | | | |
| As score increases | 2.47 | 1.42-4.30 | 0.00139 |
| Risk Optimal | | | |
| High | 1.00 | | |
| Low | 0.24 | 0.07-0.74 | 0.0137 |
| Clinical Variables: | | | |
| Lymph node infiltration | | | |
| 0 | 1.00 | | |
| 1 | 2.41 | 1.38-4.22 | 0.00198 |
| Histological grade | | | |
| High | 1.00 | | |
| Low&Intermediate | 0.65 | 0.35-1.21 | 0.173 |
| Tumor size | | | |
| A | 1.00 | | |
| B | 0.89 | 0.35-2.22 | 0.797 |
| Age | | | |
| A | 1.00 | | |
| B | 0.83 | 0.48-1.45 | 0.52 |

| | Multivariate Analysis (OSE42568 & GSE21653) | | |
|---|---|---|---|
| | Hazard Ratio | 99% CI | P value |
| Number of patients | | | |
| n = 130 | | | |
| Risk Score: | | | |
| Continous | 1.00 | | |
| As score increase | 2.30 | 1.31-4.04 | 0.00384 |
| Clinical Variables: | | | |
| Lymph node infiltration | | | |
| 0 | 1.00 | | |
| 1 | 2.19 | 1.25-3.83 | 0.00619 |

Finally, using overall survival as a primary endpoint, the risk index of the present invention was verified by the METABRIC cohort. Because of the wealth of clinical information, including adjuvant chemotherapy, we were able to select only patients who did not receive adjuvant chemotherapy, as we did in the discovery set. A total of 370 patients in the METABRIC cohort were analyzed by our risk index model. However, since only three genes (TRAT1, IL21R and CTLA4) among the five genes constituting the risk index model of the present invention were found in the METABRIC data set, and as a result, excluding 2 genes (IGHM and IL2RB), coefficients for the three genes were obtained from the METABRIC dataset, and Cox coefficient values were changed and applied using these coefficients. In the changed result, cox regression values were newly obtained and applied. β_{TRAT1} was calculated as −0.6414, β_{IL21R} was −0.2797, β_{CTLA4} was −0.3790, and F was calculated as 0.6208.

TABLE 14

| | Cox Regression estimate | Cox Regression |
|---|---|---|
| Gene | 95% confidence interval | point estimate |
| TRAT1 | −1.06659, −0.2163024 | −0.6414 |
| IL21R | −0.5429339, −0.01642154 | −0.2797 |
| CTLA4 | −0.5934638, −0.1644545 | −0.3790 |
| Lymph node status | 0.311146, 0.9303696 | 0.6208 |

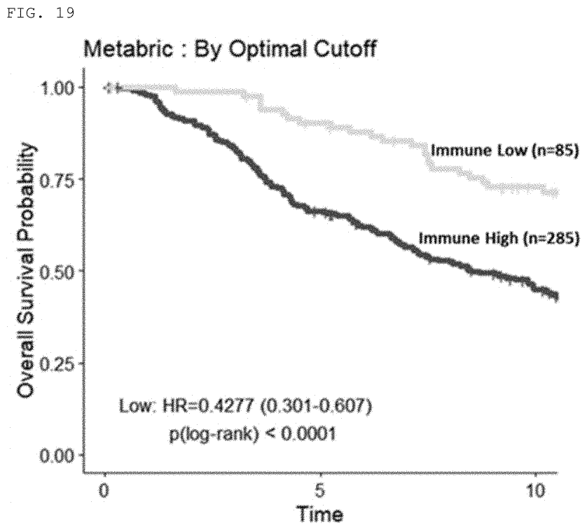

As a result of the survival analysis performed in the METABRIC cohort, the risk index model of the present invention classified by the optimal cutoff point preserved statistical significance (Table 14). As shown in FIG. 19, the Cox regression analysis performed on the METABRIC validation set confirmed that there was significant statistical significance in the immune low-risk group and high-risk group classified according to the optimal cutoff value based on the risk index model of the present invention (FIG. 19).

TABLE 15

| | Hazard Ratio | 99% CI | P value |
|---|---|---|---|
| | Univariate Analysis | | |
| Number of patients n = 370 | | | |
| Events = 250 | | | |
| Risk Score: | | | |
| Continous | | | |
| As score increase | 1.7 | 1.36-2.13 | <0.0001 |
| Risk Optimal | | | |
| High | 1.00 | | |
| Low | 0.43 | 0.30-0.61 | <0.0001 |
| Clinical Variables: | | | |
| Lymph node infiltration | | | |
| 0 | 1.00 | | |
| 1 | 1.86 | 1.37-2.53 | <0.0001 |
| Histological grade | | | |
| High | 1.00 | | |
| Low&Intermediate | 1.14 | 0.88-1.50 | 0.3080 |
| Tumor size | | | |
| A | 1.00 | | |
| B | 1.74 | 1.32-2.30 | 0.0001 |
| Age | | | |
| A | 1.00 | | |
| B | 0.86 | 0.56-1.32 | 0.4890 |
| | Multivaraite Analysis | | |
| Number of patients n = 370 | | | |
| Events = 250 | | | |
| Risk Score: | | | |
| High | 1.00 | | |
| Low | 0.50 | 0.35-0.73 | 0.0003 |
| Clinical Variables: | | | |
| Lymph node infiltration | | | |
| 0 | 1.00 | | |
| 1 | 1.22 | 0.84-1.77 | 0.2886 |
| Tumor size | | | |
| A | 1.00 | | |
| B | 1.40 | 1.02-1.92 | 0.0361 |
| Age | | | |
| A | 1.00 | | |
| B | 0.99 | 0.64-1.33 | 0.9994 |

In addition, in the METABRIC data set, the risk index of the present invention showed significance for OS (overall survival), and showed the strongest prognostic performance even after adjusting for other variables (see Table 15). The 5-year survival rate was 97.0% in the low-risk group and 72.1% in the high-risk group. The 10-year survival rate was 83.3% in the low-risk group and 51.2% in the high-risk group.

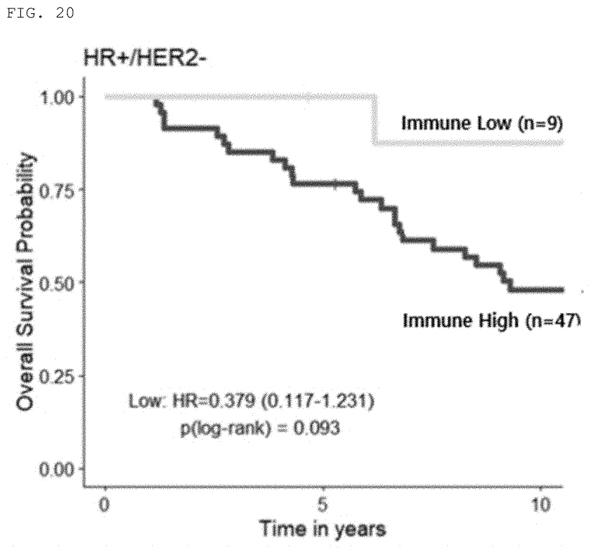

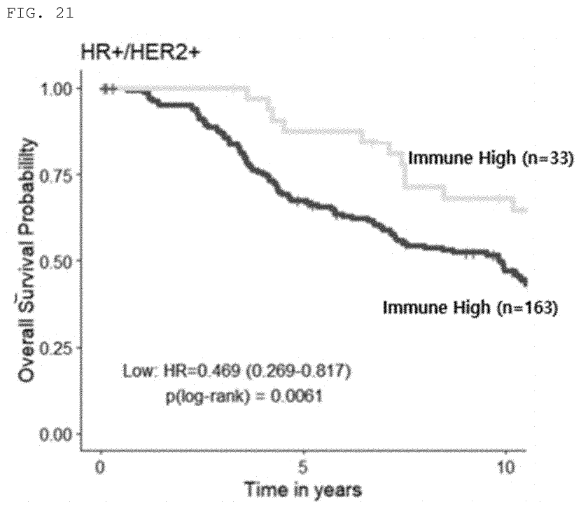

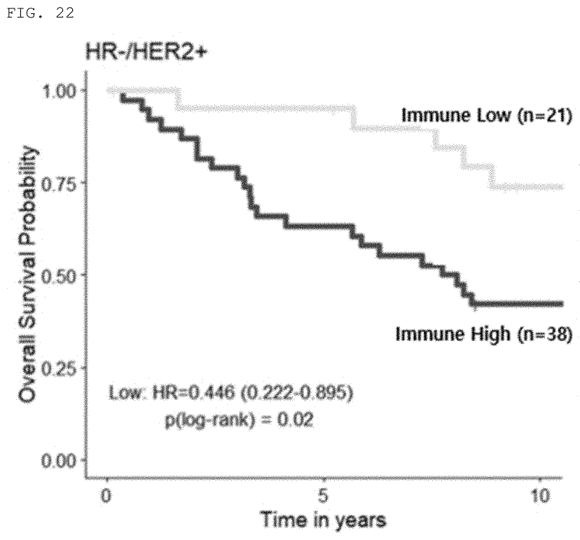

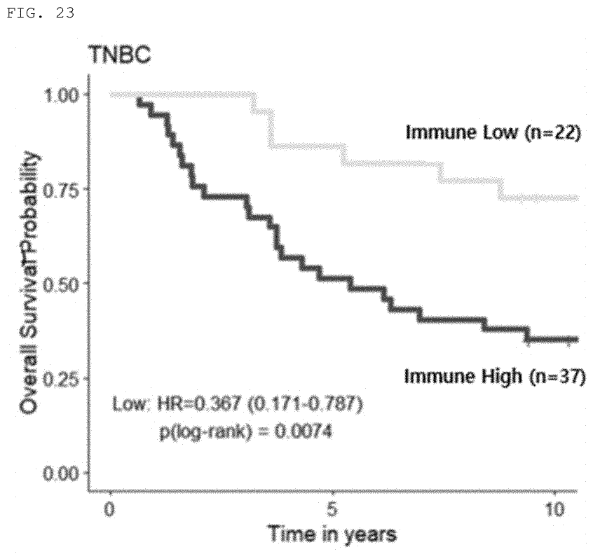

Finally, the risk index model of the present invention was applied to all breast cancer subtypes HR+/HER2−, HR=/Her2+, HR−/HER2+, and TNBC in the METABRIC data set, and the results are shown in FIGS. 20, 21, 22, and 23. As shown in FIG. 11 to FIG. 23, significance was shown in all breast cancer subtypes when the risk index model of the present invention was applied.

### Example 6: Comparative Evaluation of Breast Cancer Prognosis Prediction Model Using C-Index

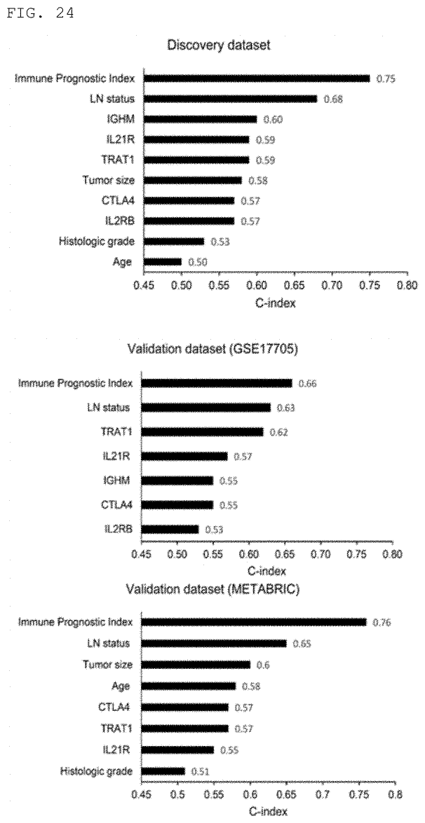

Using Harrell's Concordance Index (C-index), the performance of the existing prognosis prediction method based on other clinical variables and the risk index model for predicting breast cancer prognosis of the present invention were compared (FIG. 24). The concordance index (C-index) was calculated from the ‘survcomp’ package in R version 3.4.3. The concordance index (C-index) is a standard measure to evaluate the performance of predictive models in survival analysis.

As shown in the result of C-index in FIG. 24, compared with traditional clinicopathological variables such as lymph node status (C-index: 0.57), tumor size (C-index: 0.56), histological grade (C-index: 0.52), age (C-index: 0.50), the risk index (also referred to as immune index) in the present invention showed the highest C-index of 0.64. These results verify the independence of the risk index in the present invention as a predictive prognostic indicator of breast cancer recurrence and metastasis, and the risk index model of the present invention has better prognostic predictive performance than existing clinical pathological variables.

### INDUSTRIAL APPLICABILITY

As described above, the present invention relates to a method for predicting the prognosis of patients' breast cancer and, more particularly, to a method for predicting the prognosis of breast cancer by combining immune-related genes. The present invention may not only be applied to all patients with breast cancer regardless of breast cancer molecular subtypes, but also predict the prognosis of patients' breast cancer without information on proliferation genes by using a combination of immune-related genes to predict the prognosis of breast cancer according to the present invention. Therefore, the present invention has great industrial applicability.

1. A method for predicting the prognosis of breast cancer comprising following steps to provide information necessary for predicting the prognosis of a patient's breast cancer:
(a) measuring the expression levels of immune-related genes from a biological sample obtained from a patient with breast cancer;
(b) standardizing the expression levels measured in step (a); and
(c) predicting the prognosis of breast cancer by combining the expression levels of the immune-related genes standardized in step (b), wherein the combined overexpression levels of the immune-related genes are predicted to indicate good prognosis of breast cancer.

2. The method of claim 1, wherein the prognosis of breast cancer is at least one selected from the group consisting of recurrence, metastasis and metastatic recurrence.

3. The method of claim 1, wherein the breast cancer is a subtype selected from the group consisting of HR+/HER2−, HR+/HER2+, HR−/HER2+ and TNBC.

4. The method of claim 1, wherein the breast cancer is early breast cancer classified as LN status 0 (when no metastasis to lymph nodes has occurred) or 1 (when metastasis to lymph nodes has occurred) according to the Tumor Node Metastasis (TNM) system.

5. The method of claim 1, wherein the immune response-related genes are at least two selected from the group consisting of TRBV20-1, CCL19, CD52, SRGN, CD3D, IGJ, HLA-DRA, LOC91316, IGF1, CYBRD1, TMC5, ALDH1A1, OGN, PDCD4, FRZB, CX3CR1, IGFBP6, GLA, LOC96610, IGLL3, ITPR1, SERPINA1, EPHX2, MFAP4, RNASET2, CCNG1, FBLN5, SORBS2, CCBL2, BTN3A2, TFAP2B, LTF, ITM2A, HLA-DPB1, HLA-DMA, RPL3, LOC100130100, FAM129A, ELOVL5, GBP2, RARRES3, GOLM1, RTN1, ICAM3, LAMA2, CXCL13, ZCCHC24, CD37, VTCN1, PYCARD, CORO1A, SH3BGRL, TPSAB1, TNFSF10, ACSF2, TGFBR2, DUSP4, ARHGDIB, TMPRSS3, DCN, LRIG1, FMOD, ZNF423, SQRDL, TPST2, CD44, MREG, GIMAP6, GJA1, IFITM3, BTG2, PIP, RPS9, HLA-DPA1, IMPDH2, TNFRSF17, C14orf139, SPRY2, XBP1, THYN1, APOD, C10orf116, VAV3, FAS, MYBPC1, CFB, TRIM22, ARID5B, PTGDS, TGFBR3, TNFAIP8, SEMA3C, TMEM135, ARHGEF3, PTGER4, ABCA8, ICAM2, HLA-DQB1, HSPA2, CD27, ARMCX1, POU2AF1, IGBP1, PDE4B, ADH1B, WLS, SUCLG2, PGR, STARD13, SORL1, ATP1B1, IFT46, SIK3, LIPT1, OMD, HBB, C3, FGL2, PECI, RAC2, PDZRN3, CXCL12, DPYD, TXNDC15, STOM, EMCN, SCGB2A2, FAM176B, HIGD1A, ACSL5, RPS24, RGS10, RAI2, CNN3, FBXW4, SEPP1, SLC44A4, MGP, ABCD3, SETBP1, APOBEC3G, LCP2, HLA-DRB1, SCUBE2, DEPDC6, RPL15, SH3BP4, MSX2, CLU, DPT, ZNF238, HBP1, GSTK1, ZBTB16, CCDC69, ALDH2, SLC1A1, ARMCX2, HMGCS2, TSPAN3, FTO, PON2, C16orf62, QDPR, LRP2, PSMB8, HCLS1, FXYD1, OAT, SLC38A1, MAOA, LPL, C10orf57, SPARCL1, ERAP2, PDGFRL, RBP4, LRRC17, LHFP, BLNK, HBA2, CST7, TRAT1, IL21R, IGHM, CTLA4, IL2RB, TNFRSF9, CTSW, CCR10, GPR18, CR2, DOCK10, GZMB, ITK, LTB, IGLJ3, IGLV1-44, AIM2, CXCL9, KIAA0125, IL2RG, CD69, CD55, TRAF3IP3, EVI2B, STAP1, KLRB1, PRKCB, GPR171, PPP1R16B, SH2D1A, TNFRSF1B, CD48, BANK1, LY9, VNN2, TCL1A, CYTIP, PTPRC, PDCD1LG2, LTA, IGHG1 and CD19.

6. The method of claim 1, wherein measuring the expression levels of the genes is meant to measure the expression levels of mRNA of the genes or the expression levels of the proteins encoded by the genes.

7. The method of claim 6, wherein measuring the expression levels of mRNA of the genes is meant to measure the expression levels by a pair of primers or probes specifically binding to the genes.

8. The method of claim 6, wherein measuring the expression levels of the proteins is meant to measure the expression levels of antibodies that specifically bind to the proteins.

9. The method of claim 1, wherein the sample is selected from the group consisting of a formalin-fixed paraffin-embedded (FFPE) sample of a tissue containing the patient's cancer cells, a fresh tissue, and a frozen tissue.

10. The method of claim 1, wherein step (c) further comprises a lymph node status in which LN status of 1 (when metastasis to the lymph node has occurred) is predicted to indicate poor prognosis of breast cancer.

11. The method of claim 1, wherein step (c) is to mathematically combine the expression values of the immune-related genes standardized in step (b) to calculate a total score, and the total score indicates the prognosis of patients' breast cancer.

12. The method of claim 11, wherein, when the number of the immune-related genes is n, the mathematical combination is performed by the following Formula 1:
Total score=(β₁*χ₁)+(β₂*χ₂)+ . . . +(βₙ*χₙ) [Formula 1]
In the above formula, Xn is the expression value of the n^{th} gene, and βₙ is the Cox Regression estimate of the n^{th} gene.

13. The method of claim 11, wherein, when the number of the immune-related genes is n, the mathematical combination is performed by the following Formula 2:
Total score={(β₁*χ₁)+(β₂*χ₂)+ . . . +(βₙ*χₙ)}+F*LN [Formula 2]
In the above formula,
χₙ is the expression value of the n^{th} gene,
βₙ is the Cox Regression estimate of the n^{th} gene,
LN is an integer indicating the presence of LN, and
F is the Cox Regression estimate for LN.

14. The method of claim 1, wherein the immune-related genes is composed of T Cell Receptor Associated Transmembrane Adaptor 1 (TRAT1), Interleukin 21 Receptor (IL21R), Immunoglobulin Heavy Constant Mu (IGHM), Cytotoxic T-Lymphocyte Associated Protein 4 (CTLA4) and Interleukin 2 Receptor Subunit Beta (IL2RB).

15. The method of claim 1, wherein the immune-related genes is composed of TRAT1, IL21R and CTLA4.

16. A method for calculating a breast cancer prognostic risk score, comprising following steps in order to provide information necessary for predicting the prognosis of a patient's breast cancer:
(i) measuring the mRNA expression levels of TRAT1, IL21R, IGHM, CTLA4 and IL2RB genes from a biological sample obtained from a patient with breast cancer and the value of LN of the patient with breast cancer;
(ii) standardizing the mRNA expression levels of the genes; and
(iii) calculating a breast cancer prognostic risk score by substituting the standardized value of step (ii) and the value of LN of step (i) into the following formula 2-1:
risk score={(β_{TRAT1}*χ_{TRAT1})+(β_{IL21R}*χ_{IL21R})+(β_{IGHM}*χ_{IGHM})+(β_{CTLA4}*χ_{CTLA4})+(β_{IL2RB}*χ_{IL2RB})}+F*2*LN. <Formula 2-1>
(In formula 2-1, x is the standardized value of the expression levels of the genes indicated by a subscript,
β_{TRAT1} is −0.567144 to −0.1952896, β_{IL21R} is −0.9759746 to −0.3412672, β_{IGHM} is −0.5428339 to −0.1855019, β_{CTLA4} is −0.7454524 to −0.2010003, and β_{IL2RB} is −1.1701.266 to −1.14698,
N is an integer indicating the presence of LN, and
F is from 0.3910642 to 1.013551).

17. A method for calculating a breast cancer prognostic risk score, comprising following steps in order to provide information necessary for predicting the prognosis of a patient's breast cancer:
(i) measuring the mRNA expression levels of TRAT1, IL21R and CTLA4 genes from a biological sample obtained from a patient with breast cancer and the value of LN of the patient with breast cancer;
(ii) standardizing the mRNA expression levels of the genes; and
(iii) calculating a breast cancer prognostic risk score by substituting the standardized value of step (ii) and the value of LN of step (i) into the following formula 2-2:
risk score={(β_{TRAT1}*χ_{TRAT1})+(β_{IL21R}*χ_{IL21R})+(β_{CTLA4}*χ_{CTLA4})+F*2*LN. <Formula 2-2>
(In formula 2-2, χ is the standardized value of the expression levels of the genes indicated by a subscript,
β_{TRAT1} is −1.06659 to −0.2163024, β_{IL21R} is −0.5429339 to −0.01642154, and β_{CTLA4} is −0.5934638 to −0.1644545,
N is an integer indicating the presence of LN, and
F is from 0.311146 to 0.9303696).

18. The method of claim 16, wherein the method for measuring the expression levels of mRNA of the genes is one selected from the group consisting of microarrays, polymerase chain reaction (PCR), RT-PCR, quantitative RT-PCR (qRT-PCR), real-time polymerase chain reaction (real-time PCR), northern blot, DNA chips and RNA chips.

19. The method of claim 17, wherein the method for measuring the expression levels of mRNA of the genes is one selected from the group consisting of microarrays, polymerase chain reaction (PCR), RT-PCR, quantitative RT-PCR (qRT-PCR), real-time polymerase chain reaction (real-time PCR), northern blot, DNA chips and RNA chips.

20. A composition for predicting the prognosis of patients' breast cancer, comprising a preparation measuring the expression levels of (i) TRAT1, IL21R, IGHM, CTLA4 and IL2RB genes; or (ii) TRAT1, IL21R and CTLA4 genes.

21. The composition of claim 20, wherein the preparation is a preparation for measuring the expression levels of mRNA of the genes; or a preparation for measuring the expression levels of the proteins encoded by the genes.

22. A kit for predicting the prognosis of patients' breast cancer, comprising the composition of claim 20.

23. Use of a preparation for measuring the expression levels of (i) TRAT1, IL21R, IGHM, CTLA4 and IL2RB genes; or (ii) TRAT1, IL21R and CTLA4 genes to prepare an agent for predicting the prognosis of patients' breast cancer.
